(19) United States
(12) Patent Application Publication
(10) Pub. No.: US 2025/0073303 A1
(43) Pub. Date: Mar. 6, 2025

(54) TDP-43 Interference Peptides, Methods, and Uses Thereof

(71) Applicant: The University of British Columbia, Vancouver (CA)

(72) Inventors: Jingyan Zhu, Vancouver (CA); Max Cynader, Vancouver (CA)

(73) Assignee: The University of British Columbia, Vancouver, BC (CA)

(21) Appl. No.: 18/727,338

(22) PCT Filed: Jan. 6, 2023
(86) PCT No.: PCT/CA2023/050010

§ 371 Date: Jul. 8, 2024

Related U.S. Application Data
(60) Provisional application No. 63/297,557, filed on Jan. 7, 2022.

(51) Int. Cl. A61K 38/16; A61P 25/14; C07K 14/00; C07K 14/005

(52) U.S. Cl. CPC A61K 38/16; A61K 38/162; A61P 25/14; C07K 14/001; C07K 14/005; C12N 2740/16322; C12N 2740/16371

(57) ABSTRACT

Disclosed is a peptide including (a) a cell membrane penetration (CMP) sequence; and (b) one or more TDP-43 phosphorylation-blocking (PB) sequences. Also disclosed is a method of reducing the phosphorylation of TDP-43 in a cell, the method including: delivering an effective amount of a peptide to the cell, wherein the peptide comprises: (a) a cell membrane penetration (CMP) sequence; and (b) a TDP-43 phosphorylation-blocking (PB) sequence. Also disclosed is a method of treating a disease or pathological condition, the method comprising administering an effective amount of a peptide to a subject in need thereof; wherein the peptide comprises: (a) a cell membrane penetration (CMP) sequence; and (b) one or more TDP-43 phosphorylation-blocking (PB) sequences. Related uses, peptides for use, vectors, polynucleotide, and pharmaceutical compositions are disclosed.

### CROSS-REFERENCE TO RELATED APPLICATION

This application claims priority to U.S. Provisional Patent Application Ser. No. 63/297,557 filed on Jan. 7, 2022 entitled TDP-43 INTERFERENCE PEPTIDES, METHODS, AND USES THEREOF, the contents of which are incorporated herein by reference.

### FIELD

This application relates generally to peptides that may have therapeutic application to various diseases, and more particularly to TDP-43 interference peptides, methods, and uses thereof.

### BACKGROUND

Frontotemporal Dementia (FTD) and Amyotrophic Lateral Sclerosis (ALS) are two devastating neurodegenerative diseases that exhibit central nervous system symptoms and eventually progress to death. The most common pathological features in both diseases are TDP-43 (phosphorylated TAR DNA binding protein 43) proteinopathies which were found in over 97% of ALS patients and 50% of patients with FTLD suggesting a pivotal role in ALS and FTLD (Mackenzie and Neumann, 2016). Recently, a novel type of dementia has been defined, called limbic-predominant age-related TDP-43 encephalopathy (LATE), also characterized by the presence of TDP-43 aggregates in limbic structures in dementing elderly patients (Nelson et al., 2019). Cytoplasmic redistribution and abnormal phosphorylation of TDP-43 are necessary features of LATE, whereas the FTLD-TDP patients are divided into four main subtypes according to the patterns of TDP-43 inclusions, distribution and cortical association. In addition, unlike the TDP-43 proteinopathies within the cortical layers of FTLD patients, TDP-43 proteinopathies can also be found in the olfactory bulb, neocortex, basal ganglia and brainstem in different stages of LATE (Mackenzie et al., 2011: Josephs et al., 2008: Josephs and Dickson, 2016: Nelson et al., 2018).

“TDP-43 proteinopathy” denotes the depletion of normal nuclear TDP-43 immunoreactivity with detergent-insoluble inclusions in the neuronal cytoplasm. TDP-43 in these inclusions can be phosphorylated, ubiquitinated, and proteolytically cleaved. Although the roles of TDP-43 aggregates in neurodegenerative diseases are still under debate, it is understood that the mislocalized TDP-43 loses part of its function in the nucleus where it normally engages in several mRNA-related processes, such as transcriptional regulation, maintaining RNA stability, facilitating mRNA transport and non-coding RNA processing. Also, the misfolded form of TDP-43 accumulating in the cytoplasm has been reported to cause repression of the global protein synthesis in neuronal cells and have a comparable effect to TDP-43 knockdown (Prpar Mihevc et al., 2016; Russo et al., 2017). Furthermore, under stress conditions, which are considered as a possible cause of and contributor to neurodegenerative diseases, TDP-43 or phosphorylated TDP-43 has been reported to co-localize with stress granule (SG) markers TIA-1/PABP-1/eIF3 or regulate stress granule dynamics in cells. Disruption of stress granule assembly by ablation of ataxin-2, a protein which is necessary for SG assembly, also reduces pathology in TDP-43 mice (Becker et al., 2017). However, the relationship between TDP-43 aggregate formation and stress granule dynamics is still not fully elucidated.

In its protein structure, TDP-43 possesses a C-terminal domain with similarity to yeast prion domains which are enriched for asparagine, glutamine, tyrosine and glycine residues. Both the full length TDP-43 and the protease cleavage-generated fragments containing this domain have been observed in patients with ALS and FTLD (Neumann et al., 2006; Arai et al., 2006). Interestingly, most of the clinical mutations giving rise to ALS or FTLD are located in this region and some mutations were found to enhance aggregation, fibril formation, and neurotoxicity (Guo et al., 2011). Since the phosphorylation at dual serine residues 409 and 410 on TDP-43 is the most consistent and robust marker of pathological TDP-43 deposition, and it is not observed in the absence of neurodegeneration, abnormally phosphorylated TDP-43 has been hypothesized to mediate TDP-43 toxicity in these neurodegenerative disease states (Neumann et al., 2009b).

Effective strategies for interrupting the phosphorylation and/or aggregation of TDP-43 may therefore have value for both future research and potential therapeutic applications.

### SUMMARY

In accordance with an aspect, there is provided a peptide comprising: (a) a cell membrane penetration (CMP) sequence; and (b) one or more TDP-43 phosphorylation-blocking (PB) sequences.

In accordance with an aspect, there is provided a peptide, or a pharmaceutically acceptable salt or derivative thereof, comprising an amino acid sequence having at least about 90% identity with the amino acid sequence of SEQ ID NO:4.

In accordance with an aspect, there is provided a peptide, or a pharmaceutically acceptable salt or derivative thereof, comprising an amino acid sequence having at least about 95% identity with the amino acid sequence of SEQ ID NO:4.

In accordance with an aspect, there is provided a peptide, or a pharmaceutically acceptable salt or derivative thereof, comprising the amino acid sequence of SEQ ID NO:4.

In accordance with an aspect, there is provided a peptide comprising: (a) a cell membrane penetration (CMP) sequence: (b) a first TDP-43 phosphorylation-blocking (PB) sequence: (c) a second TDP-43 phosphorylation-blocking (PB) sequence; and (d) optionally, a linker sequence.

In accordance with an aspect, there is provided a peptide, or a pharmaceutically acceptable salt or derivative thereof, comprising an amino acid sequence having at least about 90% identity with the amino acid sequence of SEQ ID NO:5.

In accordance with an aspect, there is provided a peptide, or a pharmaceutically acceptable salt or derivative thereof, comprising an amino acid sequence having at least about 95% identity with the amino acid sequence of SEQ ID NO:5.

In accordance with an aspect, there is provided a peptide, or a pharmaceutically acceptable salt or derivative thereof, comprising the amino acid sequence of SEQ ID NO:5.

In accordance with an aspect, there is provided a peptide for use in reducing phosphorylation of TDP-43.

In accordance with an aspect, there is provided a peptide for use in reducing aggregation of TDP-43.

In accordance with an aspect, there is provided a peptide for use in reducing the formation of stress granules.

In accordance with an aspect, there is provided a peptide for use in reducing or interfering with an interaction between TDP-43 and CK18.

In accordance with an aspect, there is provided a method of reducing the phosphorylation of TDP-43 in a cell, the method comprising: delivering an effective amount of a peptide to the cell, wherein the peptide comprises: (a) a cell membrane penetration (CMP) sequence; and (b) a TDP-43 phosphorylation-blocking (PB) sequence.

In accordance with an aspect, there is provided a method of treating a disease or pathological condition, the method comprising administering an effective amount of a peptide to a subject in need thereof: wherein the peptide comprises: (a) a cell membrane penetration (CMP) sequence; and (b) one or more TDP-43 phosphorylation-blocking (PB) sequences.

In accordance with an aspect, there is provided a vector encoding the peptide.

In accordance with an aspect, there is provided a polynucleotide encoding the peptide.

In accordance with an aspect, there is provided a pharmaceutical composition comprising: an effective amount of the peptide or a pharmaceutically acceptable salt or derivative thereof; and at least one pharmaceutically acceptable excipient or adjuvant.

In accordance with an aspect, there is provided other peptides, vectors, polynucleotides, pharmaceutical compositions, uses of each thereof and of those summarized above, and methods.

### BRIEF DESCRIPTION OF THE FIGURES

Embodiments will now be described more fully with reference to the accompany drawings, in which:

FIG. 1A shows immunoblot analysis of phosphoTDP-43, TDP-43, FLAG and actin levels in the soluble and insoluble fractions of cells with either empty vector or pcDNA3.1-FLAG-CK1δ 1-317 transfection for 48 hours:

FIG. 1B shows immunoblot analysis of phosphoTDP-43, TDP-43, FLAG levels in the nuclear and cytoplasmic fractions of cells with either empty vector or pcDNA3.1-FLAG-CK1δ 1-317 transfection for 48 hours:

FIG. 1C shows confocal microscope images of human HEK293 cells with empty vector or pcDNA3.1-FLAG-CK1δ 1-317 transfection revealing increased aggregates in cells:

FIG. 2A shows confocal microscope images indicate cellular locations of TDP-43 wildtype, TDP-43 nuclear localization signal (NLS) or TDP-43 nuclear export signal (NES) with GFP fusion (Green) proteins in human HEK293 cells counterstained with DAPI (Blue):

FIG. 2B shows immunoblot analysis of soluble and insoluble proteins of HEK293 cells transfected with different expressing vectors:

FIG. 3A shows a CK-18 array membrane probed with antibody only as the negative control (Left) and probed with TDP-43 protein (Right) showing the positive binding sequences:

FIG. 3B shows the blocking effects of different peptides on TDP-43 phosphorylation:

FIG. 3C shows the specificity of peptide candidates for blocking substrates phosphorylation was confirmed by analyzing p53 protein levels in the same condition:

FIG. 3D shows the blocking effects of peptide mixture (A+B+C+D) on TDP-43 phosphorylation in soluble and insoluble fraction:

FIG. 4A shows a schematic of 29 CK1δ phosphorylation sites in the TDP-43 protein sequence:

FIG. 4B shows an immunoblot analysis of the solubility of TDP-43 variant with indicated point mutation:

FIG. 4C shows that the solubility of TDP-43 with T25A, S403A, S409A, S410A, or S409 410A mutations were quantified and the average percentage of each mutation expressed in insoluble fraction compared with wildtype were indicated:

FIG. 4D shows a schematic of peptide designed covering TDP-43 phosphorylation sites:

FIG. 4E shows the blocking effects of designed peptides at 20 μM or 40 μM analyzed in HEK293 cells with pcDNA3.1-FLAG-CK1δ1-317 transfection:

FIG. 5A shows confocal microscope images of SH-SY5Y cells with 100 μM ethacrynic acid (EA) treatment for 24 hours showed cytoplasmic localization of phosphorylated TDP-43 (Green):

FIG. 5B shows CK-18 was responsible for TDP-43 solubility in SH-SY5Y cells with 100 μM ethacrynic acid treatment:

FIG. 5C shows representative immunoblots showing effective blocking of TDP-43 phosphorylation and aggregation by 20 μM peptide mixture in SH-SY5Y cells, but not in HEK293 cells with ethacrynic acid (EA) treatment:

FIG. 5D shows dose response of peptide mixture on blocking TDP-43 aggregation in primary cultured neurons treating with 20 μM ethacrynic acid:

FIG. 5E shows the protective effects of peptide mixture on SH-SY5Y cells with ethacrynic acid challenging determined by MTT assay:

FIG. 5F shows quantification of LDH release in peptide mixture protected primary cultured neurons challenging with 20 μM ethacrynic acid:

FIG. 5G shows peptide D and mixture reduces TDP-43 phosphorylation and aggregation in SH-SY5Y cells with sodium arsenite treatment:

FIG. 5H shows peptide D reduces stress granule numbers in SH-SY5Y cells with sodium arsenite treatment via confocal microscope images of SH-SY5Y cells with scramble or peptide D treatment after 1 mM sodium arsenite challenging for 1 hour:

FIG. 6A shows confocal images of immunohistochemical TDP-43 (Green) or phosphoTDP-43 (409/410) (Red) staining of ipsilateral and contralateral lumbar spinal cord slices 3 days after sciatic nerve axotomy:

FIG. 6B shows immunoblots demonstrating TDP-43, phosphoTDP-43 (409/410) and actin levels in ipsilateral lumbar cord tissue (control group vs peptide group) in the soluble and insoluble fraction at indicated days after sciatic nerve axotomy:

FIG. 6C shows a comparison of scramble peptide and peptide mixture group showing TDP-43 and actin levels in ipsilateral lumbar spinal cord tissue in the soluble and insoluble fraction on day 3, 4 and 5 after sciatic nerve axotomy. * p<0.05, ** p<0.01:

FIG. 6D shows confocal images of immunohistochemical TDP-43 (Green) or phosphoTDP-43 (409/410) (Red) staining of ipsilateral lumbar spinal cord slices 3 days after sciatic nerve axotomy in scramble peptide and peptide mixture group:

FIG. 7A shows immunoblotting of HSP70 and GAPDH proteins in ipsilateral lumbar cord tissue (control group vs peptide group) at the indicated days after sciatic nerve axotomy:

FIG. 7B shows confocal images of immunohistochemical HSP70 staining of ipsilateral lumbar spinal cord slices in both groups showed nuclear translocation on days 1, 2 and 3 after sciatic nerve axotomy:

FIG. 8A shows confocal images of immunohistochemical TIA-1 (Red) staining of ipsilateral lumbar spinal cord slices in control group or peptide mixture group at indicated days after sciatic nerve axotomy:

FIG. 8B shows immunoblotting of TIA-1 and GAPDH proteins of control group or peptide mixture group in the soluble fraction and insoluble fraction of ipsilateral lumbar spinal cord tissue at the indicated days after sciatic nerve axotomy:

FIG. 8C shows comparison of TIA-1 expression in the insoluble fraction between control group and peptide mixture group. * p<0.05. ** p<0.01:

FIG. 9A shows immunoblot analysis of phosphoTDP-43, TDP-43 and actin in HEK293 cells which were transiently transfected with empty vector, pCS2-Myc-CK1δ and pcDNA3.1-FLAG-CK1δ1-317 after 48 hours:

FIG. 9B shows immunoblot analysis of phosphoTDP-43, TDP-43, FLAG and actin in HEK293 cells expressing empty vector or pcDNA3.1-FLAG-CK1δ 1-317 at 24 hours, 48 hours and 72 hours after transfection:

FIG. 10 shows coimmunoprecipitation and immunoblotting of SH-SY5Y cell (Left) or HEK293 cell (Right) lysate transiently expressing pcDNA3.1-FLAG-CK1δ 1-317:

FIG. 11 shows HEK293 cells were transfected with pcDNA3.1-FLAG-CK1δ1-317 for 48 hours, with the different combinations of peptide candidates at concentration of 5 μM, 10 μM, 15 μM, 20 μM and 25 μM being added into the cultured cells, the protein levels of phosphoTDP-43 (409/410) and TDP-43 were detected by western blotting:

FIG. 12 shows Western blot analysis of protein expression (TDP-43, phosphoTDP-43, PARP and actin) in either soluble or insoluble fractions of HL-1 cells with 16 h treatment of sodium arsenite (0.05 mM):

FIG. 13 shows Western blot analysis of TDP-43 and phosphoTDP-43 expression in the soluble and insoluble fraction of ipsilateral lumbar spinal cord tissue that was collected in mice three days post LPS (5 mg/kg) plus sciatic nerve axotomy procedure:

FIG. 14 shows Fluoro Jade (neurodegeneration marker) staining of ventral horn of spinal cord of mice in each indicated treatment group (upper panel) and counterstained with DAPI (lower panel):

FIG. 15 shows Iba-1 staining of lumbar spinal cord of mice in each indicated group were counterstained with DAPI, where 20 mg/kg of peptide D was injected daily through the i.p. route:

FIG. 16 shows markers of (A) ChAT staining (red) indicated motor neurons and (B) NeuN staining (green) counterstained with DAPI indicated alpha motor neurons in ventral horn of mice in each group; and

FIG. 17 shows TDP-43 aggregation in lumbar spinal cord evaluated by Western blotting.

### DETAILED DESCRIPTION OF EMBODIMENTS

The present description relates to peptides that can reduce TDP-43 phosphorylation and/or aggregation and which may have therapeutic application to various diseases, including neurodegenerative diseases such as amyotrophic lateral sclerosis (ALS) and frontotemporal dementia (FTD).

The present inventors have found that overexpression of Casein Kinase 18 (CK-18) in HEK293 cells causes phosphorylation and reduced solubility of endogenous TDP-43, and that such CK-18-caused TDP-43 phosphorylation is related to TDP-43 aggregation which is the common pathological feature of various neurodegenerative diseases. The inventors have further found that certain peptides as described herein can interfere with the interaction between CK-18 and TDP-43, thereby reducing or preventing this phosphorylation and/or aggregation of TDP-43.

The present inventors have further found that interfering peptides derived from CK-18 binding domains or from TDP-43 phosphorylation sites can block TDP-43 phosphorylation induced by CK-18 overexpression, including in in vitro and in vivo models of cell stress and cell death. For example, in the in vitro studies described herein, a mixture of peptides according to the present description reduced ethacrynic acid-induced TDP-43 phosphorylation and aggregation as well as cell death in both SH-SY5Y cells and in primary cultured neurons. In a mouse in vivo axotomy model, characterized by a reliable time-specific pattern of TDP-43 phosphorylation and aggregate formation, treatment with peptides according to the present description efficiently prevented the development of TDP-43 phosphorylation and aggregation in ventral horn motor neurons of the lumbar spinal cord.

The present inventors have also found that peptides according to the present description can reduce stress granule formation without affecting HSP70 nuclear translocation.

Taken together, and without wishing to be bound by any particular theory, these results suggest that blocking TDP-43 phosphorylation by the use of peptides as described herein may provide a useful therapeutic strategy, for example, by targeting aggregates that form in several neurodegenerative diseases.

The present description provides, in one aspect, a membrane-permeable peptide or mixture of peptides which reduces or prevents the phosphorylation of TDP-43.

In one aspect, the peptide reduces or prevents the aggregation of TDP-43.

In one aspect, the peptide reduces or prevents the formation of stress granules.

In one aspect, a peptide according to the present description comprises a cell membrane penetrating domain and at least one TDP-43 phosphorylation-blocking sequence.

In one aspect, the peptide comprises: (a) a cell membrane penetrating domain: (b) a first TDP-43 phosphorylation-blocking sequence: (c) a second TDP-43 phosphorylation-blocking sequence; and (d) optionally, a linker sequence.

In one aspect, a blocking sequence of the peptide is derived from a sequence of CK-18.

In one aspect, a blocking sequence of the peptide is derived from a sequence of TDP-43.

In one aspect, the peptide selectively blocks the phosphorylation of TDP-43, without significantly affecting other CK1δ substrates.

In another aspect, the present description relates to a method of reducing the phosphorylation of TDP-43 in a cell, the method comprising: delivering an effective amount of a peptide to the cell, wherein the peptide comprises: (a) a cell membrane penetration (CMP) sequence; and (b) a TDP-43 phosphorylation-blocking (PB) sequence.

In one aspect, the delivery of the peptide to the cell may be conducted in vitro, or ex vivo, or in vivo.

In another aspect, the present description relates to a method of treating a disease or pathological condition comprising administering an effective amount of a peptide comprising: (a) a cell membrane penetration (CMP) sequence; and (b) one or more TDP-43 phosphorylation-blocking (PB) sequences, to a subject in need thereof.

In one aspect, the subject may be a human.

In one aspect, the disease or pathological condition may be associated with the phosphorylation and/or aggregation of TDP-43.

In one aspect, the disease or pathological condition may be associated with the formation or presence of stress granules.

In one aspect, the disease or pathological condition may be a neurodegenerative disease.

In one aspect, the neurodegenerative disease may be ALS, FTD, or LATE.

In one aspect, the peptide may be administered systemically, e.g., intravenously or subcutaneously.

In another aspect, the present description relates to a vector or polynucleotide encoding a peptide as described herein.

In another aspect, the present description relates to a pharmaceutical composition comprising: (a) an effective amount of a peptide as described herein, or a pharmaceutically acceptable salt or derivative thereof; and (b) at least one pharmaceutically acceptable excipient or adjuvant.

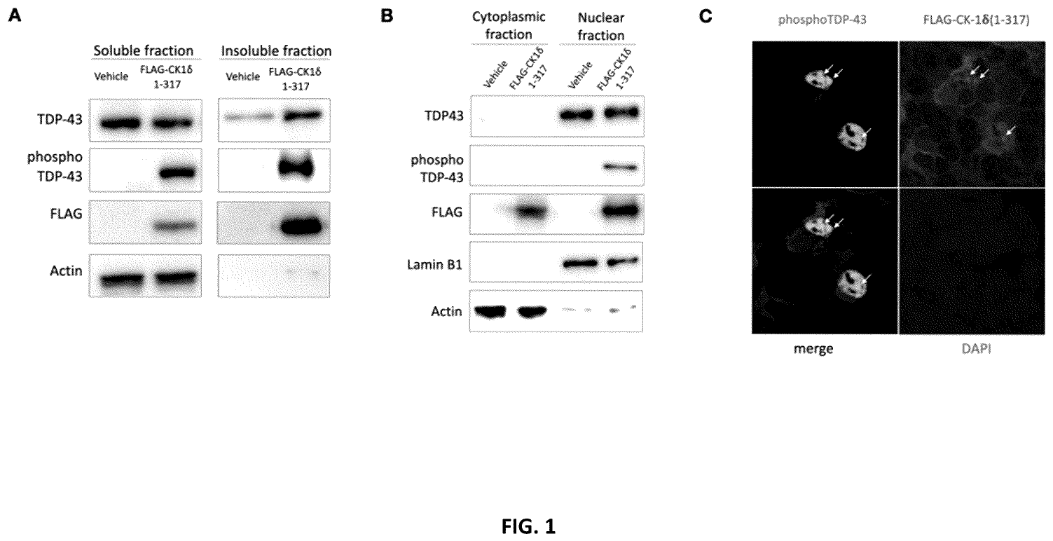

FIG. 1. The hyperactive form of CK1δ (CK1δ 1-317) causes TDP-43 phosphorylation and aggregation with a predominant nuclear location in HEK293 cells. (A). Immunoblot analysis of phosphoTDP-43, TDP-43, FLAG and actin levels in the soluble and insoluble fractions of cells with either empty vector or pcDNA3.1-FLAG-CK1δ 1-317 transfection for 48 hours. (B) Immunoblot analysis of phosphoTDP-43, TDP-43, FLAG levels in the nuclear and cytoplasmic fractions of cells with either empty vector or pcDNA3.1-FLAG-CK1δ 1-317 transfection for 48 hours. Lamin B1 and actin were used as nucleus and cytoplasmic markers. (C) Confocal microscope images of human HEK293 cells with empty vector or pcDNA3.1-FLAG-CK1δ 1-317 transfection revealing increased aggregates in cells. The cells were immunostained with anti-phosphorylated TDP-43 (pS409/410) polyclonal antibody (Green), anti-FLAG monoclonal antibody (Red) and were counterstained with DAPI (Blue). Arrows indicate the aggregates.

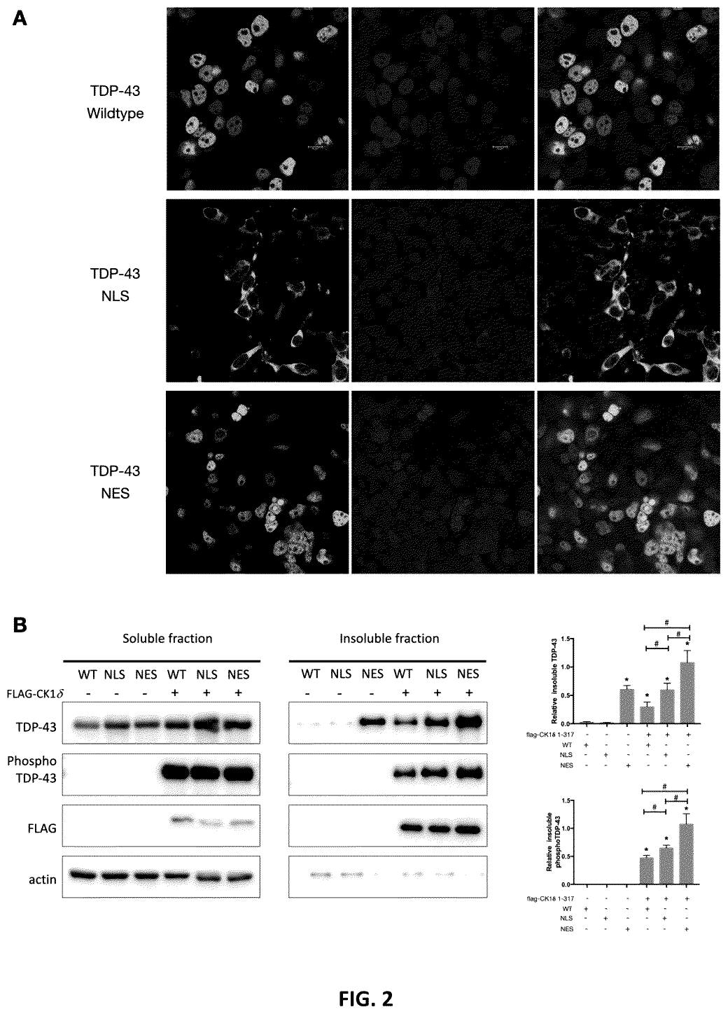

FIG. 2. Disruption of TDP-43 nuclear-cytoplasmic transportation is associated with decreased solubility. (A) Confocal microscope images indicate cellular locations of TDP-43 wildtype, TDP-43 nuclear localization signal (NLS) or TDP-43 nuclear export signal (NES) with GFP fusion (Green) proteins in human HEK293 cells counterstained with DAPI (Blue). (B) Immunoblot analysis of soluble and insoluble proteins of HEK293 cells transfected with different expressing vectors. TDP-43, phosphoTDP-43, FLAG and actin were probed as indicated. Quantifications indicate that the majority of TDP-43 ANES was in the insoluble fraction. The solubility of TDP-43 wildtype, ANLS and ANES were all decreased with CK1δ (1-317) co-transfection.

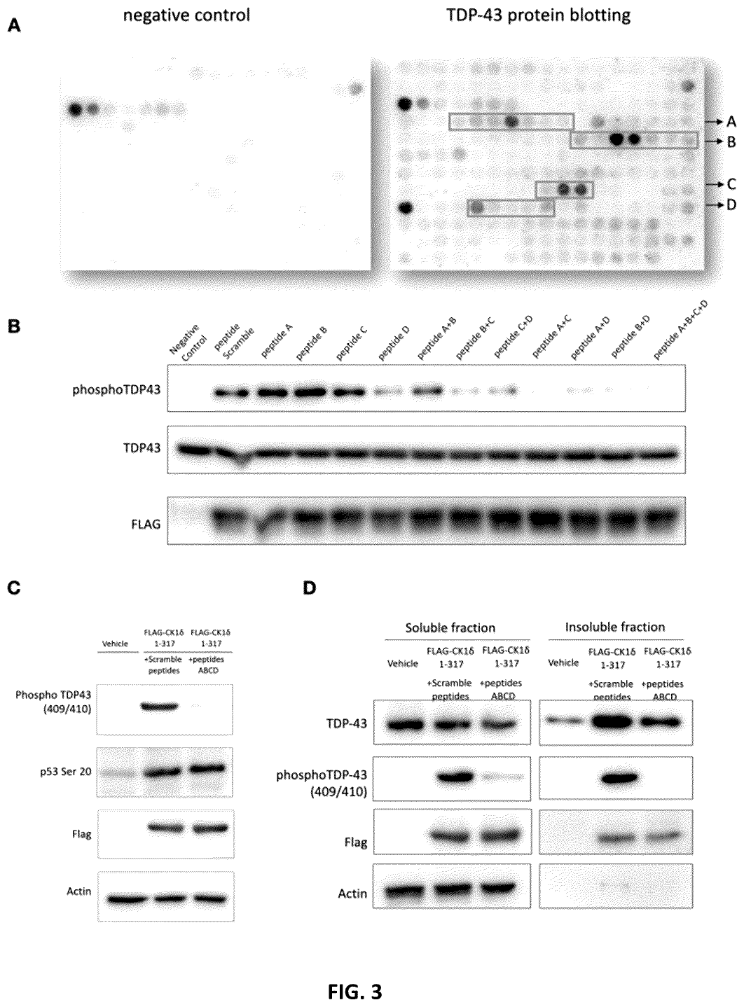

FIG. 3. Synthetic peptides derived from CK1δ block TDP-43 phosphorylation and partially reduce TDP-43 aggregation caused by CK-1δ overexpression. (A) CK-1δ array membrane was probed with antibody only as the negative control (Left) and probed with TDP-43 protein (Right) showing the positive binding sequences. Four peptide candidates (A, B, C and D) were designed from overlapped sequences of consecutive positive spots. The array membrane was stripped and the experiment was repeated three times. (B) The blocking effects of different peptides on TDP-43 phosphorylation. HEK293 cells were transfected with pcDNA3.1-FLAG-CK1δ1-317 plasmids for 48 hours and were treated with a final concentration of 20 μM peptide candidates with different combinations as indicated. The protein levels of phosphoTDP-43 (409/410), TDP-43 and FLAG were detected by western blotting. (C) The specificity of peptide candidates for blocking substrates phosphorylation was confirmed by analyzing p53 protein levels in the same condition. (D) The blocking effects of peptide mixture (A+B+C+D) on TDP-43 phosphorylation in soluble and insoluble fraction. A total of 20 μM (5 μM of each peptide) peptide mixture was added to HEK293 cells expressing pcDNA3.1-FLAG-CK1δ1-317 and the protein levels of TDP-43, phosphoTDP-43 (409/410), FLAG and Actin in the soluble and insoluble fractions were analyzed by western blotting.

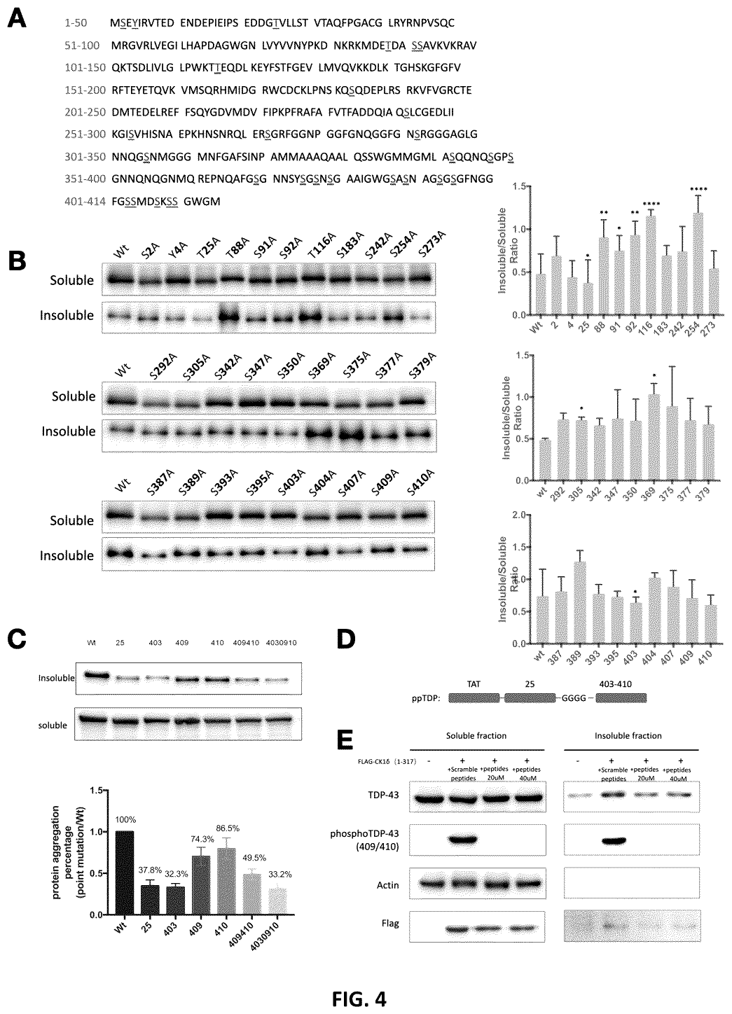

FIG. 4. Peptide designed from TDP-43 phosphorylation sites blocks TDP-43 phosphorylation and partially blocks TDP-43 aggregation with CK1δ overexpression. (A) Schematic of 29 CK1δ phosphorylation sites in the TDP-43 protein sequence. (B) Immunoblot analysis of the solubility of TDP-43 variant with indicated point mutation. TDP-43 in the soluble and insoluble fractions was quantified. n=3, *p<0.05, **p<0.01 and ****p<0.0001. (C) The solubility of TDP-43 with T25A, S403A, S409A, S410A, or S409 410A mutations were quantified and the average percentage of each mutation expressed in insoluble fraction compared with wildtype were indicated. (D) Schematic of peptide designed covering TDP-43 phosphorylation sites. (E) The blocking effects of designed peptides at 20 μM or 40 μM were analyzed in HEK293 cells with pcDNA3.1-FLAG-CK1δ1-317 transfection. The expression of TDP-43, phosphoTDP-43, actin and Flag in the soluble and insoluble fraction were detected by western blotting.

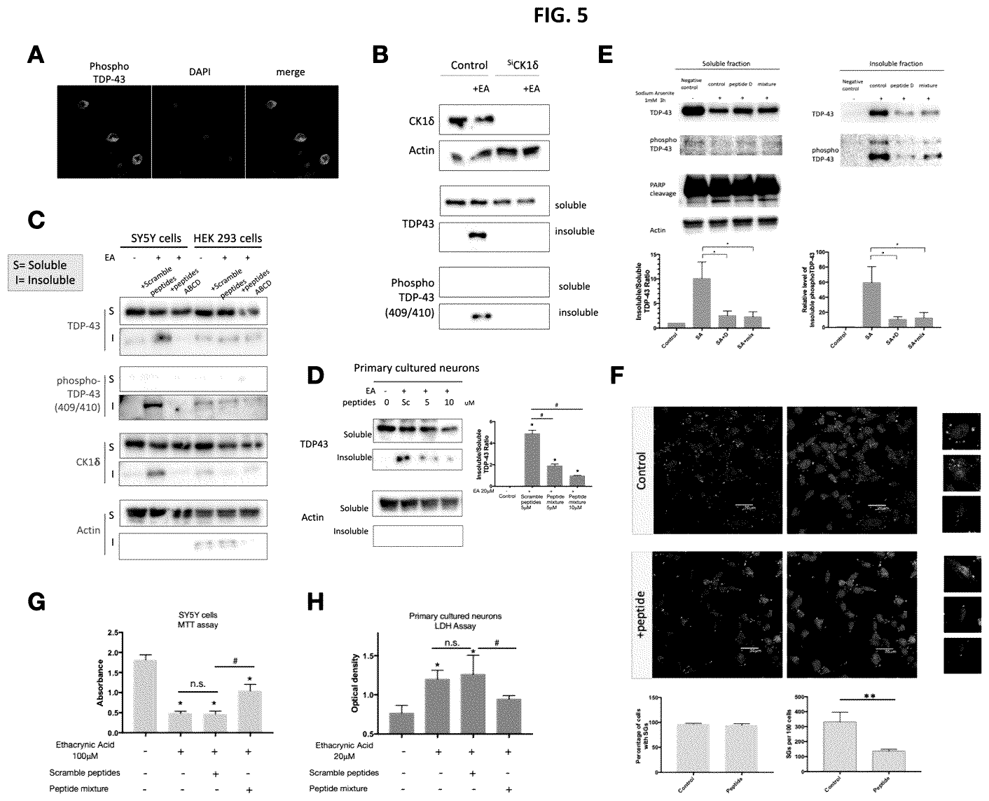

FIG. 5. Synthetic peptides derived from CK-1δ block TDP-43 phosphorylation, aggregation and partially rescue cell death in different cellular models. (A) Confocal microscope images of SH-SY5Y cells with 100 μM ethacrynic acid (EA) treatment for 24 hours showed cytoplasmic localization of phosphorylated TDP-43 (Green). DAPI (Blue) staining was used to determine nuclear localization. (B) CK-1δ was responsible for regulation of TDP-43 solubility in SH-SY5Y cells with 100 μM ethacrynic acid treatment. Representative immunoblots showing efficient siRNA knockdown of CK-1δ in SH-SY5Y cells. TDP-43 and phosphoTDP-43 were absent in the insoluble fraction with knock down of CK-1δ after ethacrynic acid treatment. (C) Representative immunoblots showing effective blocking of TDP-43 phosphorylation and aggregation by 20 μM peptide mixture in SH-SY5Y cells, but not in HEK293 cells with ethacrynic acid (EA) treatment. (D) Dose response of peptide mixture on blocking TDP-43 aggregation in primary cultured neurons treated with 20 μM ethacrynic acid. Quantification of insoluble TDP-43 expression was shown on the right. (E) The protective effects of peptide mixture on SH-SY5Y cells with ethacrynic acid challenge were determined by MTT assay. The significance among the groups was calculated by one-way ANOVA, *p<0.05. The statistical significance of the difference between scramble peptides and peptide mixture groups was calculated by student's t test, #p<0.05. (F) Quantification of LDH release in peptide mixture protected primary cultured neurons challenged with 20 μM ethacrynic acid. Significant differences among the groups was calculated by One-way ANOVA, *p<0.05. Significance between scramble peptides and peptide mixture group was calculated by student's t test, #p<0.05. (G) Peptide D and the peptide mixture reduces TDP-43 phosphorylation and aggregation in SH-SY5Y cells following sodium arsenite treatment. SH-SY5Y cells in four groups (Negative control, scramble control, peptide D, peptide mixture) were treated with or without 1 mM sodium arsenite for 3 hours. TDP-43, phosphoTDP-43, PARP, and actin in the soluble and insoluble fraction were analyzed by western blotting. Quantifications of insoluble TDP-43 and phosphoTDP-43 expression are shown below. *p<0.05 (H) Peptide D reduces stress granule numbers in SH-SY5Y cells with sodium arsenite treatment. Confocal microscope images of SH-SY5Y cells with scramble or peptide D treatment after 1 mM sodium arsenite challenge for 1 hour. Cells were stained for the stress granule marker TIA-1 (Green) and DAPI (Blue). Representative cells in each group are shown on the right. Bar graphs demonstrated the percentage of cells with SGs and SG numbers per 100 cells. **p<0.01. Scale bar 20 μm.

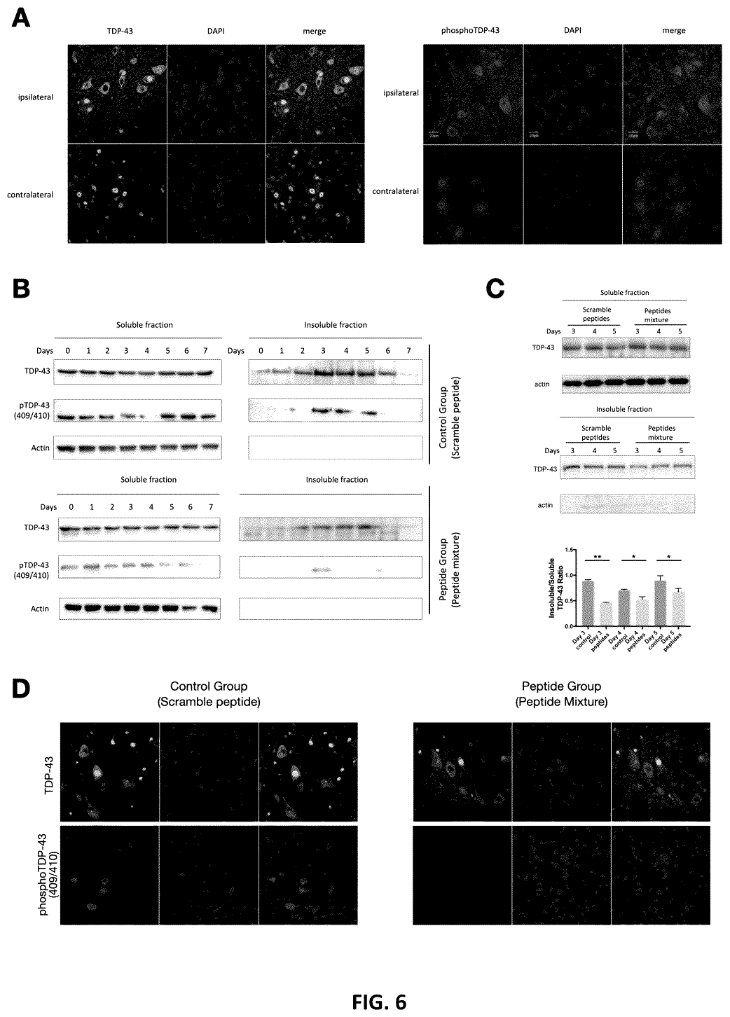

FIG. 6. Synthetic peptides derived from CK-1δ block TDP-43 phosphorylation and reduce TDP-43 aggregation in vivo. (A) Confocal images of immunohistochemical TDP-43 (Green) or phosphoTDP-43 (409/410) (Red) staining of ipsilateral and contralateral lumbar spinal cord slices 3 days after sciatic nerve axotomy. The slices were counterstained with DAPI (blue). Scale bar 20 μm. (B) Immunoblots demonstrating TDP-43, phosphoTDP-43 (409/410) and actin levels in ipsilateral lumbar cord tissue (control group vs peptide group) in the soluble and insoluble fraction at indicated days after sciatic nerve axotomy. The peptide mixture reduced TDP-43 and phosphoTDP-43 (409/410) accumulation in the insoluble fraction on days 3, 4 and 5 post-axotomy. (C) Comparison of scrambled peptide and peptide mixture group showing TDP-43 and actin levels in ipsilateral lumbar spinal cord tissue in the soluble and insoluble fraction on day 3, 4 and 5 after sciatic nerve axotomy. *p<0.05, **p<0.01. (D) Confocal images of immunohistochemical TDP-43 (Green) or phosphoTDP-43 (409/410) (Red) staining of ipsilateral lumbar spinal cord slices 3 days after sciatic nerve axotomy in scramble peptide and peptide mixture group. The slices were counterstained with DAPI (blue). Scale bar 20 μm.

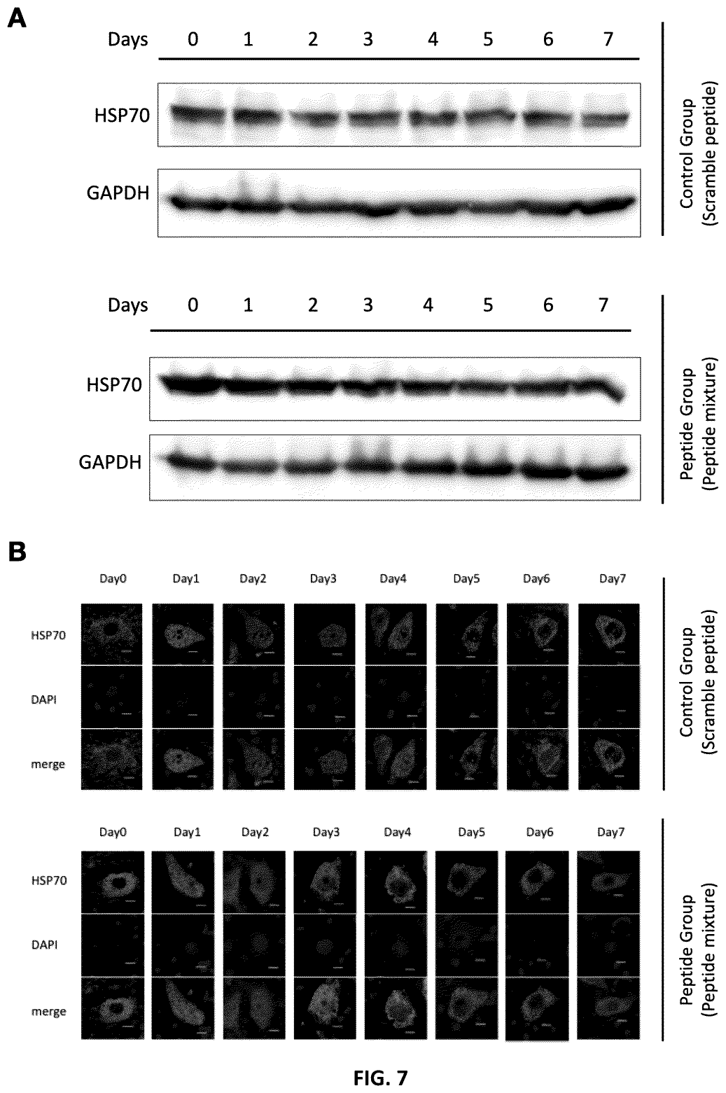

FIG. 7. Treatment with synthetic peptides derived from CK-1δ does not affect HSP70 activity in ventral horn motor neurons after sciatic nerve axotomy. Peptide mixture administration once per day in sciatic nerve axotomy mice. (A) Immunoblotting of HSP70 and GAPDH proteins in ipsilateral lumbar cord tissue (control group vs peptide group) at the indicated days after sciatic nerve axotomy. The levels of HSP70 in both groups were not regulated by sciatic nerve axotomy. (B) Confocal images of immunohistochemical HSP70 staining of ipsilateral lumbar spinal cord slices in both groups showed nuclear translocation on days 1, 2 and 3 after sciatic nerve axotomy. Scale bar, 5 m.

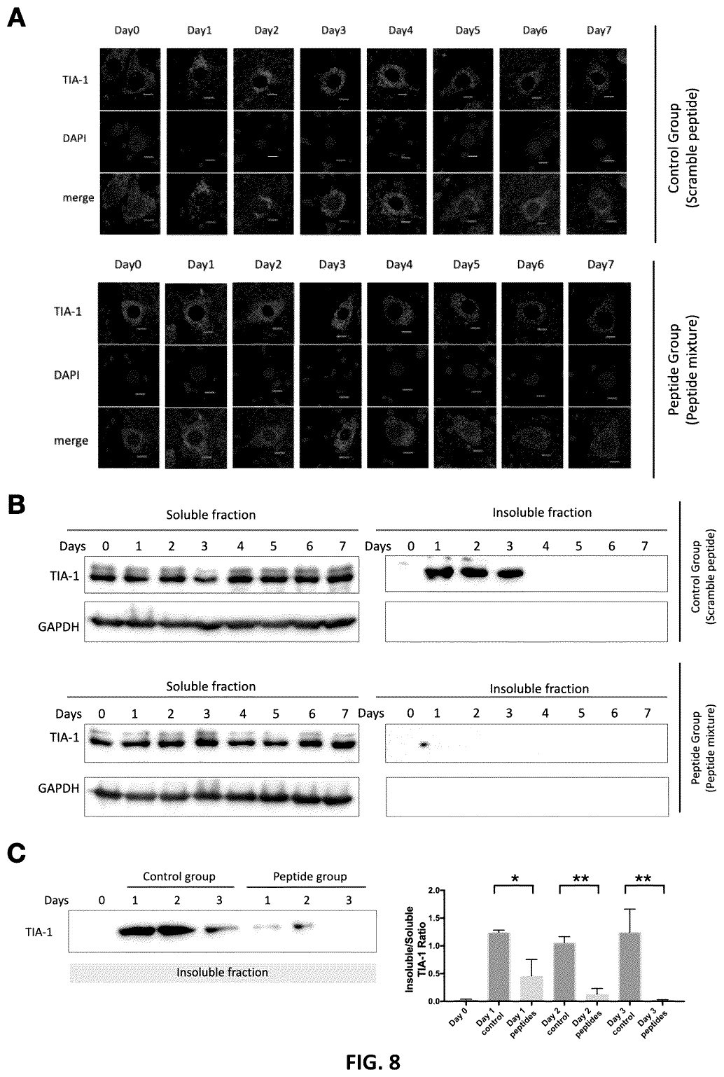

FIG. 8. Treatment with synthetic peptides derived from CK-1δ reduces stress granule formation in ventral horn motor neurons after sciatic nerve axotomy. Scrambled peptides or the peptide mixture were administered once a day to sciatic nerve axotomy mice from day 0 to 7 post-surgery. (A) Confocal images of immunohistochemical TIA-1 (Red) staining of ipsilateral lumbar spinal cord slices in control group or peptide mixture group at indicated days after sciatic nerve axotomy. TIA-1 was concentrated as aggregates in cytoplasm on days 1, 2 and 3 after sciatic nerve axotomy. Scale bar, 5 m. (B) Immunoblotting of TIA-1 and GAPDH proteins of control group or peptide mixture group in the soluble fraction and insoluble fraction of ipsilateral lumbar spinal cord tissue at the indicated days after sciatic nerve axotomy. TIA-1 was increased in the insoluble fraction on days 1, 2 and 3 after sciatic nerve axotomy and this was not found in peptide-treated group. (C) Comparison of TIA-1 expression in the insoluble fraction between the control group and the peptide mixture group. *p<0.05, **p<0.01.

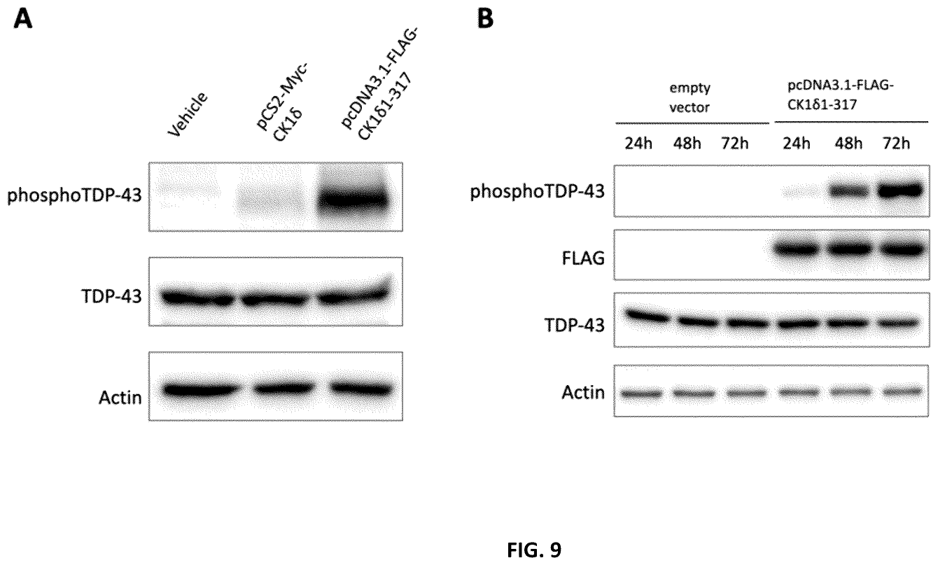

FIG. 9. TDP-43 phosphorylation by the hyperactive form of CK-1δ occurs in a time dependent manner. (A) Immunoblot analysis of phosphoTDP-43, TDP-43 and actin in HEK293 cells which were transiently transfected with empty vector, pCS2-Myc-CK1δ and pcDNA3.1-FLAG-CK1δ1-317 examined after 48 hours. (B) Immunoblot analysis of phosphoTDP-43, TDP-43, FLAG and actin in HEK293 cells expressing empty vector or pcDNA3.1-FLAG-CK1δ 1-317 assessed at 24 hours, 48 hours and 72 hours after transfection.

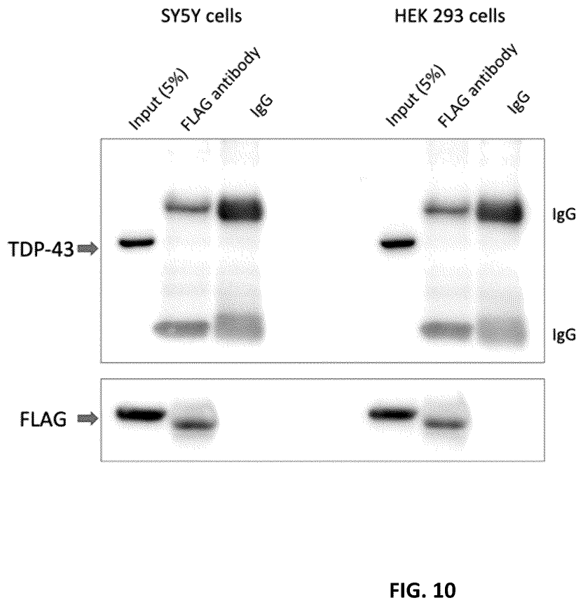

FIG. 10. TDP-43 does not stably bind with CK1δ. Co-immunoprecipitation and immunoblotting of SH-SY5Y cell (Left) or HEK293 cell (Right) lysate transiently expressing pcDNA3.1-FLAG-CK1δ 1-317. FLAG antibody was used to pull down proteins and the immunoblots were probed with a TDP-43 antibody and a FLAG antibody. TDP-43 was not pulled down with FLAG-CK1δ 1-317 in cell lysates.

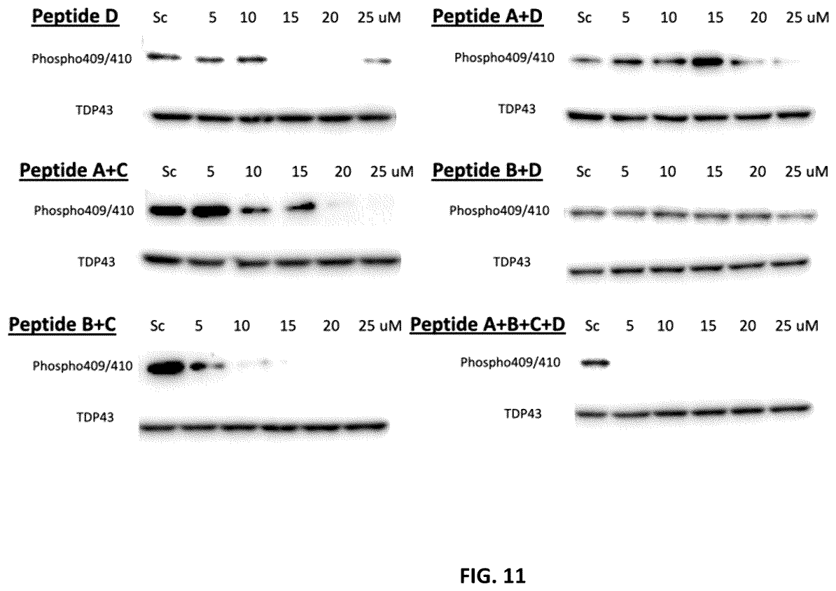

FIG. 11. The dose effects of different combinations of peptide candidates on blocking TDP-43 phosphorylation. HEK293 cells were transfected with pcDNA3.1-FLAG-CK1δ1-317 for 48 hours. The different combinations of peptide candidates at concentration of 5 μM, 10 μM, 15 μM, 20 μM and 25 μM were added into the cultured cells. The protein levels of phosphoTDP-43 (409/410) and TDP-43 were detected by western blotting.

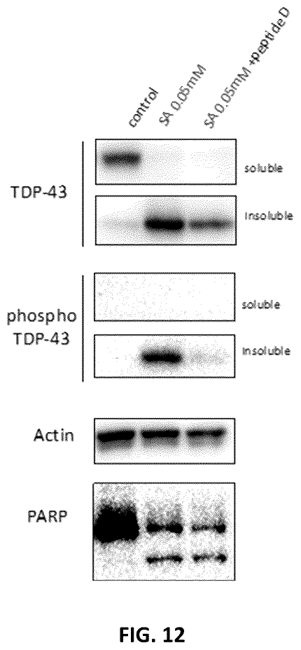

FIG. 12. Example peptide D reduces TDP-43 and phosphoTDP43 aggregates in HL-1 cells following sodium arsenite treatment. Western blot analysis of protein expression (TDP-43, phosphoTDP-43, PARP and actin) in either soluble or insoluble fractions of HL-1 cells with 16 h treatment of sodium arsenite (0.05 mM). 20 μM of peptide D efficiently reduced TDP-43 and phosphoTDP-43 aggregates in sodium arsenite treated HL-1 cells.

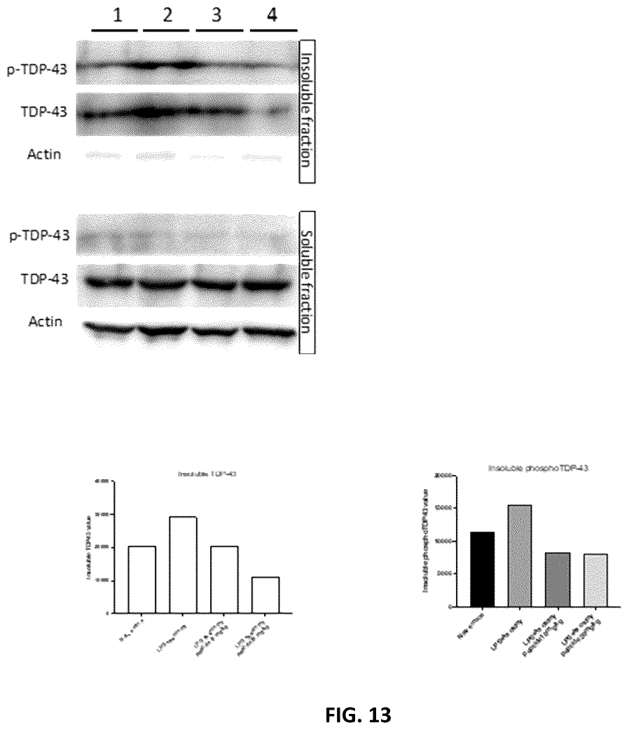

FIG. 13: Example peptide D dose dependently reduces TDP-43 and phosphoTDP-43 aggregates in lumbar spinal cord of mice with LPS plus sciatic nerve axotomy. Western blot analysis of TDP-43 and phosphoTDP-43 expression in the soluble and insoluble fraction of ipsilateral lumbar spinal cord tissue that was collected in mice three days post LPS (5 mg/kg) plus sciatic nerve axotomy procedure. Group 1: Non-surgical mice. Group 2: Control mice with LPS plus sciatic nerve axotomy. Group 3: 10 mg/kg of peptide D treatment (once daily, i.p.) in mice with LPS treatment plus sciatic nerve axotomy. Group 4: 20 mg/kg of peptide D treatment (once daily, i.p.) in mice with LPS treatment plus sciatic nerve axotomy.

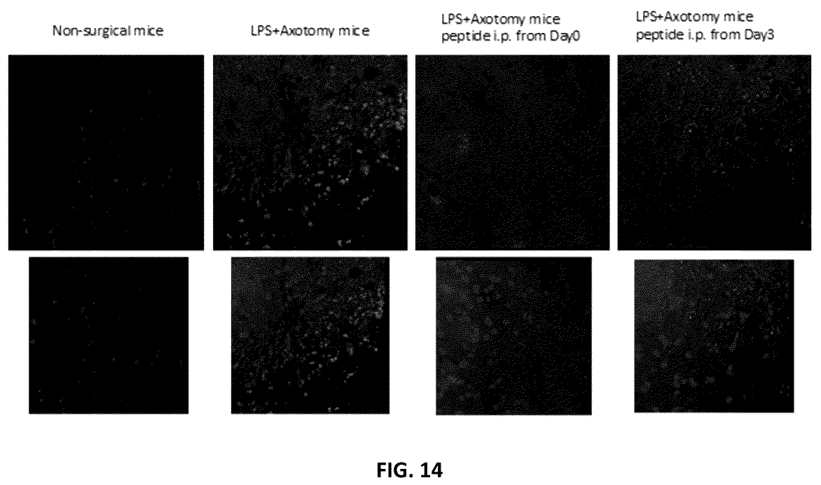

FIG. 14: Example peptide D prevents neurodegeneration in ventral horn of spinal cord of mice with LPS plus sciatic nerve axotomy. Mice underwent LPS administration (5 mg/kg, i.p.) plus sciatic nerve axotomy on day 0 and tissues were collected and fixed on day 7 after the surgical procedure. Fluoro Jade (neurodegeneration marker) staining of ventral horn of spinal cord of mice in each indicated treatment group (upper panel) and counterstained with DAPI (lower panel). 20 mg/kg of peptide D was injected daily through i.p. route.

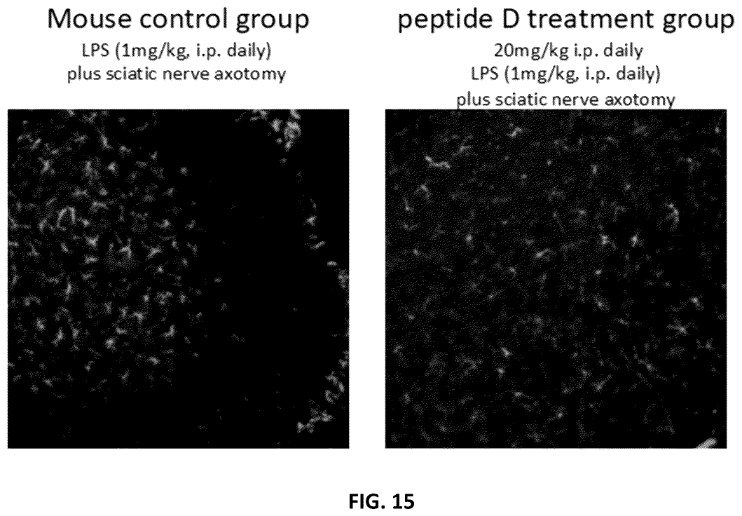

FIG. 15: Example peptide D reduces microglia activation in lumbar spinal cord of mice with LPS plus sciatic nerve axotomy. Mice underwent LPS administration (1 mg/kg, daily, i.p.) plus sciatic nerve axotomy on day 0 and tissues were collected and fixed on day 7 post-surgery. Iba-1 staining of lumbar spinal cord of mice in each indicated group were counterstained with DAPI. 20 mg/kg of peptide D was injected daily through the i.p. route.

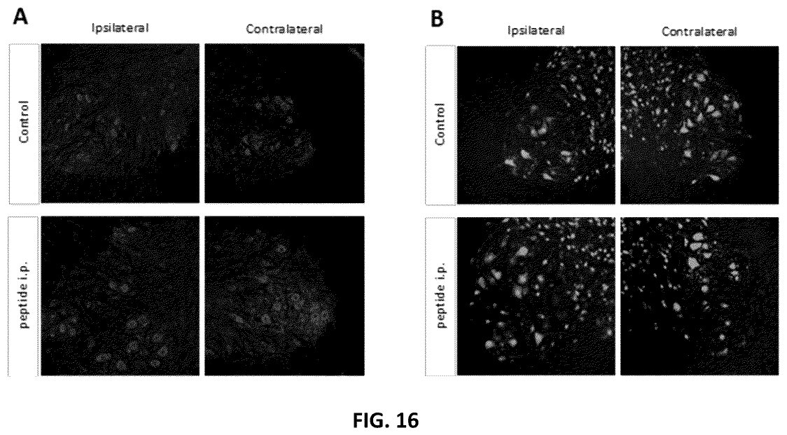

FIG. 16: Example peptide D prevents motor neuron death in ventral horn of spinal cord (L6) of mice with LPS plus sciatic nerve axotomy. Mice underwent LPS treatment (1 mg/kg, daily, i.p.) plus sciatic nerve axotomy on day 0 and tissue was collected and fixed on day 7 post-surgery. Markers of (A) ChAT staining (red) indicating motor neurons and (B) NeuN staining (green) counterstained with DAPI indicated alpha motor neurons in ventral horn of mice in each group. The number of motor neurons in contralateral ventral horn were used as control to compare motor neuron loss in ipsilateral ventral horn spinal cord vs contralateral (control) ventral horn after combined LPS treatment and sciatic nerve axotomy.

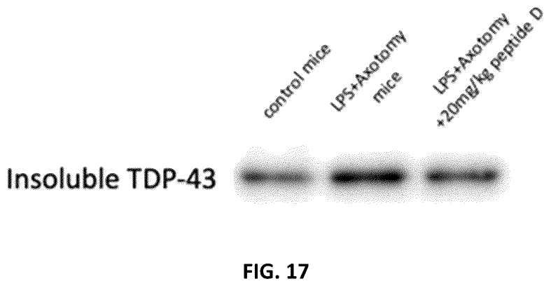

FIG. 17: Example peptide D reduces TDP-43 aggregates in lumbar spinal cord of mice with sciatic nerve axotomy and 7 days of LPS administration. Mice underwent sciatic nerve axotomy (day 0) and LPS (1 mg/kg, daily, i.p.) administration for 7 days (days 0-7). TDP-43 aggregation in lumbar spinal cord was evaluated by western blotting. Mice treated with peptide D (20 mg/kg i.p.) showed less TDP-43 aggregation in lumbar spinal cord when compared with that of control LPS+Axotomy mice.

### EXAMPLES

### Methods and Materials

Cell lines and mouse primary cortical neuron culture: Human cell lines: Human Embryonic Kidney 293 (HEK293) and human neuroblastoma (SH-SY5Y) cells were purchased from American Type Culture Collection (ATCC). Cells were maintained in Dulbecco's modified Eagle's medium (DMEM; Sigma Chemical Co., St. Louis, MO) and supplemented with 10% fetal bovine serum (FBS) (GIBCO BRL, Grand Island, NY) and 1% antibiotics (Gibco-BRL, Grand Island, NY). All cultures were maintained at 37° C. in a humidified incubator (NuAir, Plymouth, MN) with 95%02 and 5% CO₂ and were passaged when they achieved 90% confluence. Primary cortical neuron cultures were prepared from the embryos of E15 timed pregnant CD1 mice. The embryos were cut and transferred to the dish with HBSS. The whole brains were then dissected and the meninges were gently peeled to open the cortical lobes. The cortices were separated and transferred to 15 mL tubes containing HBSS. After a gentle spin, the HBSS was replaced with 5 mL of 0.25% trypsin and tissue was incubated in a 37° C. water bath for 20 minutes to digest the tissue. Then, the cortical tissues were triturated using a 10 mL pipette and washed twice with DMEM containing 10% Fetal Bovine Serum. After spinning at 1500 rpm for 5 minutes, the cell pellet was resuspended in 5-10 mL neurobasal plating medium (Gibco-BRL, Grand Island, NY) supplemented with 2% B27 (Gibco-BRL, Grand Island, NY), 2 mM L-glutamine (Sigma, Saint Louis, MO), 25 μM glutamic acid (Sigma, Saint Louis, MO), 10 mM β-mercaptoethanol (Gibco-BRL, Grand Island, NY) and 1% penicillin-streptomycin (Sigma, Saint Louis, MO). These neuronal cells were then plated and cultured on poly-D-lysine coated 6-well plates at a density of 8×10⁵ cells per well.

The medium was replaced on the second day and afterwards half of the medium was replaced every 3-4 days. The cultures were maintained at 37° C. in a humidified incubator with 5% CO₂.

Cell treatment and peptide blocking assay: Ethacrynic acid (EA) (Sigma, Saint Louis, MO) was dissolved in dimethyl sulfoxide (DMSO) at a stock concentration of 100 mM. For SH-SY5Y cells and HEK-293 cells, ethacrynic acid (EA) was added to the culture medium at 1:1000 to reach a final concentration of 100 μM. The treatment was maintained for 24 hours. For mouse cortical neurons, mature cortical neurons of 14 days in vitro (DIV) were used for experiments. Ethacrynic acid (EA) was added to neurons at a concentration of 20 μM for 5 hours. For peptide treatment in cell culture, the peptides were dissolved in double distilled water at a stock concentration of 10 mM and stored at −80° C. The combination of peptides A, B, C and D at equal concentration of 5 μM for each peptide and 20 μM in total was added into cultures of SH-SY5Y cells or HEK-293 cells one hour before ethacrynic acid treatment or pcDNA3.1-FLAG-CK1δ1-317 transfection. The primary cultured neurons were treated with peptide A, B, C and D mixture (equal concentration for each) at a final concentration of 5 μM and 10 μM.

Plasmids, siRNAs and transfection: The expression vectors for HEK293 and SH-SY5Y cells used in this study were as follows: pCS2-Myc-CK1δ, pcDNA3.1-FLAG-CK1δ1-317, pEGFP-C1-TDP-43 wildtype, pEGFP-C1-TDP-43 ΔNLS and pEGFP-C1-TDP-43 ΔNES. pCS2-Myc-CK1δ and pcDNA3.1-FLAG-CK1δ1-317 plasmids were the generous gift of Dr. Takashi Nonaka and detailed information about plasmid construction is described in Nonaka et al., 2016. EGFP-C1-TDP-43 wildtype was created by cloning TDP-43 full length sequences into a pEGFP-Clvector using BamH1 and Xba1 digestion sites. The primers for generating EGFP-C1-TDP-43 wildtype are: forward 5′ CGCGGATCCCGCATGTCTGAATATATT 3′, reverse 5′ GCTCTAGAGCCTACATTCCCCAGCCAG 3′. TDP-43 carrying mutations in putative nuclear localization signals (NLS) and nuclear export signals (NES) were generated using the Q5 site-directed mutagenesis kit (NEB, US) using EGFP-C1-TDP-43 wildtype as the template. For pEGFP-C1-TDP-43 ANLS, the first three amino acids of the nuclear localization signals of TDP-43 were changed: K82A, R83A, K84A. For pEGFP-C1-TDP-43 ANES, the last three amino acids of nuclear export signals of TDP-43 were changed: L248A, I249A, I250A. The primers used in the site-directed mutagenesis experiments are TDP-43 ANLS Forward: 5′-AGCAATGGATGAGACAGATGCTTC-3′, TDP-43 ΔNLS Reverse: 5′-GCTGCGTTATCTTTTGGATAGTTGACAAC-3′; TDP-43 ΔNES Forward: 5′-AGCAAAAGGAATCAGCGTTCATATATC-3′, TDP-43 ΔNES Reverse: 5′-GCTGCGTCCTCTCCACAAAGAGAC-3′. The PCR amplification, KLD reaction and transformation followed the protocol procedure. The 29 predicted phosphorylation sites in the TDP-43 sequences were mutated to Alanine using the Q5 site-directed mutagenesis kit (NEB, US) using pEGFP-C1-TDP-43 wildtype as the template. The primers used in the site-directed mutagenesis experiments are: S2A forward: 5′ATCCCGCATGgctGAATATATTCGG 3′, reverse: 5′ CCCCAGCCAGAAGACTTA3′; Y4A forward: 5′ CATGTCTGAAgctATTCGGGTAACCGAAG 3′, reverse: 5′ TACATTCCCCAGCCAGAA 3′; T25S forward: 5′ AGACGATGGGgctGTGCTGCTCT 3′, reverse: 5′ TCCGATGGTATTTCAATGGG 3′; T88A forward: 5′ AATGGATGAGgctGATGCTTCATC 3′, reverse: 5′ TTTCTTTTGTTATCTTTTGGATAG 3′; S91A forward: 5′ GACAGATGCTgctTCAGCAGTGAAAG 3′, reverse: 5′ TCATCCATTTTTCTTTTGTTATCTTTTG 3′; S92A forward: 5′ AGATGCTTCAgctGCAGTGAAAG 3′, reverse: 5′ GTCTCATCCATTTTTCTTTTG 3′; Ti 16A forward: 5′ ATGGAAAACAgctGAACAGGACC 3′, reverse: 5′ GGGAGACCCAACACTATTAAATC 3′; S183A forward: 5′ TTCTAAGCAAgctCAAGATGAGCCTTTG3′, reverse: 5′ TTAGGAAGTTTGCAGTCAC 3′; S242A forward: 5′ GATTGCGCAGgctCTTTGTGGAG 3′, reverse: 5′ TGATCATCTGCAAATGTAACAAAGG 3′; S254A forward: 5′ TAAAGGAATCgctGTTCATATATCCAATGCC 3′, reverse: 5′ ATGATCAAGTCCTCTCCAC 3′; S273A forward: 5′ GTTAGAAAGAgctGGAAGATTTGGTGGTAATC 3′, reverse: 5′ TGTCTATTGCTATTGTGC 3′; S292A forward: 5′ ATTTGGTAATgctAGAGGGGGTGGAG 3′, reverse: 5′ CCACCCTGATTCCCAAAG 3′; S305A forward: 5′ CAATCAAGGTgctAATATGGGTGGTGGGATG 3′, reverse: 5′ TTTCCCAAACCAGCTCCA 3′; S342A forward: 5′ CATGTTAGCCgctCAGCAGAACCAGTC 3′; reverse: 5′ CCCATCATACCCCAACTG 3′; S347A forward: 5′ GCAGAACCAGgctGGCCCATCGG 3′, reverse: 5′ TGGCTGGCTAACATGCCC 3′; S350A forward: 5′ GTCAGGCCCAgcIGGTAATAACCAAAAC 3′, reverse: 5′ TGGTTCTGCTGGCTGGCT 3′; S369A forward: 5′ GGCCTTCGGTgctGGAAATAACTC 3′, reverse: 5′ TGGTTTGGCTCCCTCTGC 3′; S375A forward: 5′ TAACTCTTATgctGGCTCTAATTCTGGTGCAG 3′, reverse: 5′ TTTCCAGAACCGAAGGCC 3′; S377A forward: 5′ TTATAGTGGCgctAATTCTGGTG 3′, reverse: 5′ GAGTTATTTCCAGAACCG 3′; S379A forward: 5′ TGGCTCTAATgctGGTGCAGCAA 3′, reverse: 5′ CTATAAGAGTTATTTCCAGAACCGAAG 3′; S387A forward: 5′ TGGTTGGGGAgctGCATCCAATG 3′, reverse: 5′ ATTGCTGCACCAGAATTAG 3′; S389A forward: 5′ GGGATCAGCAgctAATGCAGGGT 3′, reverse: 5′ CAACCAATTGCTGCACCAG 3′; S393A forward: 5′ CAATGCAGGGgctGGCAGTGGTT 3′, reverse: 5′ GATGCTGATCCCCAACCAATTG 3′; S395A forward: 5′ AGGGTCGGGCgctGGTTTTAATGGAG 3′, reverse: 5′GCATTGGATGCTGATCCC3′; S403A forward: 5′AGGCTTTGGCgctAGCATGGATT3′, reverse: 5′CCATTAAAACCACTGCCC3′; S404A forward: 5′ CTTTGGCTCAgctATGGATTCTAAGTC 3′, reverse: 5′ CCTCCATTAAAACCACTG3′; S407A forward: 5′ AAGCATGGATgctAAGTCTTCTG 3′, reverse: 5′ GAGCCAAAGCCTCCATTA 3′; S409A forward: 5′ GGATTCTAAGgctTCTGGCTGGG 3′, reverse: 5′ ATGCTTGAGCCAAAGCCTC3′; S410A forward: 5′ TTCTAAGTCTgctGGCTGGGGAA 3′, reverse: 5′ TCCATGCTTGAGCCAAAG 3′.

The plasmids were transfected into HEK293 or SH-SY5Y cells using the Lipofectamine 2000 system. Before transfection, cells were plated at 80˜90% confluence and cultured in DMEM containing 10% fetal bovine serum (FBS). DNA and lipofectamine reagent were mixed at a 1:2.5 ratio in opti-MEM Reduced Serum Medium for 20 minutes and then added to cells. After 4-5 hours, the transfection medium was changed to fresh DMEM containing 10% fetal bovine serum. The pre-designed silencing siRNA was purchased from Invitrogen (siRNA ID: 146134). The sequences (5′-3′) are: sense: CCGAUGAGAACUCUCCUUATT; Antisense: UAAGGAGAGUUCUCAUCGGTG. The pre-designed siRNA was transfected into cells using Lipofectamine RNAiMAX reagent according to the manufacturer's instructions.

Immunoprecipitation-Mass Spectrometry: For the antibody-beads coupling preparation: 50 μl NHS-activated sepharose beads were washed with 500 μl cold 1 mL HCl followed by rapid washing with 500 μl coupling buffer (0.2M NaHCO₃, 0.5M NaCl, pH 8.3), repeated 3 times. Then, mixed beads were incubated with 5 μl antibody (phosphoTDP-43 409/410 or TDP-43) in 20 μl coupling buffer and rotated at 4° C. overnight. On the second day, coupled beads were washed with 500 μl coupling buffer 2 times, followed by blocking in Tris-buffer (0.1M Tris-HCl, pH 8.5) for 2 hours at room temperature. Then, beads were washed alternatively with Tris buffer and acetate buffer (0.1M Acetic acid, 0.1 M Sodium acetate, 0.5M NaCl, pH 4.5) 3 times on ice. The coupled beads were stored in 20% ethanol until used. The cell lysates were prepared from HEK293 cells transfected with either empty vector or pcDNA3.1-FLAG-CK1δ1-317 for 48 hours. Cells were lysed in Lysis buffer (20 mM Tris-HCl pH 7.5, 50 mM NaCl, 1% NP-40, 3 mM MgCl₂, 1 mM CaCl₂), 1mM Na₃VO₄, 10 mM NaF, 50 mM Na₄P₂O₇) on ice for 10 minutes. The supernatant was collected after spinning at 16,000×g for 10 minutes and then precleared with goat anti-mouse IgG agarose (Sigma). After that, the cell lysate was incubated with coupled beads, rocked at 4° C. for 2 hours and then washed with 1 ml lysis buffer. The protein complexes were eluted from beads using 6M urea/2M thiourea in 10 mM Tris-HCl pH8.0 at room temperature for 30 minutes. Each protein sample was mixed with 4× sample buffer and run in SDS-PAGE gel. The proteins digested from the gel were subjected to mass spectrometry analysis.

Protein-protein coimmunoprecipitation assay: 48 hours after transfection with pcDNA3.1-FLAG-CK1δ1-317 plasmid, cells were washed with PBS twice and harvested on ice with gentle lysis buffer (25 mM Tris-Hcl, 10 mM NaCl, 20 mM EDTA, 10 mM EGTA, 0.5% Triton-100, 10% Glycerol, 1 mM dithiothreitol and protease inhibitor) for 10 minutes. Then, the cell lysate was sonicated and centrifuged at 16,000×g for 10 minutes. The supernatant was further incubated with 10 ul 50% protein A/G magnetic beads (Themo, Rockford, IL) for 30 minutes to remove nonspecific binding. Then, the FLAG antibody was added into the supernatant and incubated at 4° C. overnight. On the second day, 50 ul 50% protein A/G magnetic beads were added into the mixture for another 2 hours. After washing with gentle lysis buffer 3 times, the proteins were eluted from the beads with 35-50 μl 2× lysis buffer and boiled for 3 minutes. The samples were then ready for further analysis by western immunoblotting.

Fractionation of cellular soluble and insoluble proteins: Cells or spinal cord tissue were harvested and lysed in 500 μl RIPA buffer [50 mM Tris-HCl pH 7.4; 150 mM NaCl; 1% NP-40; 0.5% Na-deoxycholate; 1 mM EDTA; 50 mM NaF; 1 mM PMSF and protease inhibitor] on ice. The lysates were sonicated and centrifuged at 20,000×g for 15 minutes at 4° C. The supernatant was recovered as a soluble fraction. The pellet was sonicated and washed in RIPA buffer for 3 times, then suspended in 200 μl UREA buffer [7M Urea; 2M Thiourea; 2% SDS, 30 mM Tris-HCl, pH 8.5 and protease inhibitor]. The pellet was sonicated and maintained at room temperature for 20 minutes, then centrifuged at 20,000×g for 10 minutes. The resulting samples were used as the insoluble fraction. All the protein samples were further analyzed by Western immunoblotting.

Nuclear and cytoplasmic protein separation: The nuclear and cytoplasmic proteins were separated using an NE-PER Nuclear and Cytoplasmic Extraction kit (Pierce, Rockford, IL). Briefly, the cells in 6 well plates were washed with PBS 3 times and completely air-dried on ice. Then, cells were lysed in 200 μl ice-cold CER I buffer for 10 minutes and softly scratched into a tube on ice. 11 μl ice-cold CER II was added to each tube and vortexed for 5 seconds. After 1 minute of incubation, the lysates were centrifuged for 5 minutes at 14,000×g. The supernatant was recovered as cytoplasmic proteins. The pellets were further lysed in ice-cold NER buffer and vortexed for 15 seconds every 10 minutes, for a total of 40 minutes. Finally, the lysates were centrifuged at 14,000×g for 10 minutes. The supernatant was collected as nuclear proteins and stored at −80° C. for further analysis.

Tissue protein extraction: After decapitation, the spinal cords were immediately removed and the ventral column of the lumbosacral cord segments of the axotomy side were collected. Tissues from 4 mice were then subjected to protein extraction and western blotting analysis. The tissues were washed in cold PBS 3 times and homogenized in RIPA buffer. After 10 minutes, the lysates were sonicated and centrifuged at 20,000×g for 15 minutes at 4° C. The soluble fraction was collected from the supernatant. The pellet was re-sonicated and lysed in the RIPA buffer again for another 10 minutes and then centrifuged at 20,000×g for 10 minutes. The pellet was sonicated and suspended in UREA buffer for 20 minutes at room temperature. The supernatant was collected as the insoluble fraction after centrifuged at 20,000×g for 10 minutes. The resulting samples were further analyzed by Western immunoblotting.

Western immunoblotting: Protein samples were mixed with 4× sample buffer and denatured by heating at 100° C. before loading into SDS-PAGE gel. Proteins in UREA buffer skipped the denaturation step of protein preparation. According to the protein concentration, equal amounts of protein in each sample were loaded into 5% stacking gels and separated in 10% SDS-polyacrylamide resolving gels using a Bio-Rad Gel electrophoresis system (Bio-Rad, Hercules, CA). PageRuler™ Plus Prestained Protein ladder (Fermentas, CA) was loaded as the molecular weight indicator for protein samples. Proteins in the SDS-PAGE gel were then transferred to a PVDF membrane using the Bio-Rad Wet Transfer System (Bio-Rad, Hercules, CA) running at 100 volts for 90 minutes at 4° C. The PVDF membrane was activated before the transfer procedure by soaking it in methanol for one minute. After proteins were transferred onto the PVDF membrane, the membrane was blocked in 5% BSA in TBST (Tris buffered saline tween 20) for one hour at room temperature, then incubated with primary antibodies at 4° C. overnight. On the second day, the membrane was washed 3×10 min in TBST and then incubated with horseradish peroxidase conjugated secondary antibody (Goat anti-rabbit IgG, PerkinElmer, NEF812001EA, 1:5000 dilution or Goat anti-mouse IgG, PerkinElmer, NEF822001EA, 1:5000 dilution) for one hour. Then, the membrane was washed again for 3×10 min in TBST and visualized in the Bio-Rad Imaging system (Bio-Rad ChemiDoc MP) using ECL Western blotting substrate. The relative density of each protein was calculated by normalization with actin or GAPDH on the same membrane using Bio-Rad Quantity One software. The primary antibodies used in the study are listed in Table 1, below.

TABLE 1

| Name | Company | Host and clonality | dilution |
|---|---|---|---|
| Anti phosphoTDP-43 | Cosmo Bio Co | Mouse Monoclonal | 1:1000 |
| (pS409/410) | | | |
| TARDBP | Proteintech | Rabbit Polyclonal | 1:1000 |
| Casein Kinase 1 Delta | abcam | Mouse Monoclonal | 1:1000 |
| Phospho-p53 (Ser20) | Cell Signaling | Rabbit Polyclonal | 1:1000 |
| Anti-FLAG M2 | Sigma | Mouse Monoclonal | 1:1000 |
| β-Actin | Cell Signaling | Rabbit Polyclonal | 1:1000 |
| Anti-Lamin B1 | abcam | Rabbit Polyclonal | 1:1000 |
| Anti-GAPDH [6C5] | abcam | Mouse Monoclonal | 1:1000 |
| Anti-Hsp70 [5A5] | abcam | Mouse Monoclonal | 1:1000 |
| Anti-TIA1 | abcam | Rabbit Monoclonal | 1:1000 |

Immunocytochemistry: The cells were plated and cultured on poly-D-lysine coated glass coverslips at a density of 5×10⁵ cells per well in a 12-well plate. At the experimental time point, cells were washed with cold PBS (pH 7.4) and fixed in 4% Paraformaldehyde (PFA) in PBS for 15 minutes. Then, cells were washed with cold PBS 2 times and permeabilized with 0.25% Triton X-100 in PBS for 10 minutes at room temperature. To block the nonspecific binding of the antibodies, cells were washed with PBS 3 times to completely remove the Triton X-100 and incubated with 1% BSA in PBST (0.1% Tween 20 in 1×PBS) for 30 minutes at room temperature. After that, the primary antibody against phosphoTDP-43 (1:250; Proteintech Rabbit polyclonal, 22309-1-AP) and FLAG (1:500; Sigma Mouse monoclonal) was added to the cells and incubated for 2 hours. Then, cells were washed with PBS 3 times to remove primary antibody residue and probed with anti-rabbit secondary antibody conjugated with Alexa 488 (Invitrogen, Carlsbad, CA) and anti-mouse secondary antibody conjugated with Alexa 568 (Invitrogen, Carlsbad, CA) for 1 hour at room temperature. The coverslips with cells were washed with PBS for 3 times and mounted on glass slides with antifade reagent with DAPI to counterstain nuclear DNA. All samples were stored at 4° C. in dark and subjected to confocal scan imaging using an Olympus Fluoview FV1000 confocal laser microscope.

Peptide Array: The peptide spot array on the cellulose-based membrane was synthesized at the UBC peptide synthesis facility using a previously published protocol (Hilpert et al., 2007). The array contained overlapping peptides (12-mer peptides with 2 amino acids shift per spot) to cover the full sequence of CK1δ. To initialize the array membrane, the membrane was washed twice with methanol for 10 minutes at room temperature followed by washing with TBST three times. Then the array membrane was ready to use. Before bait protein binding, the membrane was blocked with 5% sucrose and 4% non-fat dry milk in TBST for 4 hours at room temperature. Then it was incubated with 10 μg/ml TDP-43 recombinant protein in 4% non-fat dry milk overnight at 4° C. or 4% non-fat dry milk only as the negative control. On the second day, the membrane was washed with TBST and incubated with rabbit polyclonal TDP-43 antibody overnight at 4° C. After that, the membrane was washed again in TBST and incubated with secondary antibody for 2 hours at room temperature. Finally, the membrane was washed with TBST and was ready to visualize the positive protein binding spots by an enhanced chemiluminescence reaction assay.

Cell-penetrating peptides design and synthesis: Peptides were designed from overlapped sequences of continuous positive binding spots on the peptide arrays. To make the peptides suitable for cell penetration, a sequence consisting of the truncated HIV-1 TAT domain was added at the N-terminal. The designed peptides were then synthesized and purified by HPLC with over 90% purity. The resulting peptides were verified by mass spectrometry and dissolved in double distilled water at stock concentration.

MTT assay: The cell viability of SH-SY5Y cells after treatment of Ethacrynic acid (EA) was determined using the MTT assay (Sigma, Saint Louis, MO). Briefly, MTT solution was added to cells for 3-4 hours at 37° C. at the time when the cell viability was determined. Then, the lysis buffer was added into each well and incubated overnight. On the second day, the plate was shaken for 15 minutes in the dark and absorbance at OD=595 nm was read using a plate reader (Envision 2103 Multilabel Reader, Perkin Elmer).

Lactate dehydrogenase (LDH) assay: The viability of primary cultured neurons after the treatment of Ethacrynic acid (EA) was determined using the Lactate dehydrogenase (LDH) assay (in vitro toxicology assay kit; Sigma, Saint Louis, MO). The release of lactate dehydrogenase was measured by incubating the cultured medium from each condition with the reagent for 1 hour in the 96 well plate according to the manufacturer's instructions. The optical density of each sample was measured at a wavelength of 490 nm and a reference filter of 750 nm using the ‘uQuant’ microplate spectrophotometer. LDH readings of each condition were subtracted from the reading of 100% survival well and then converted to the percentage of neuronal death by comparing with the reading of 100% death well which was incubated with 1% triton-X100 for 10 minutes.

Immunohistochemistry: Before the mouse spinal cord was assessed using immunohistochemistry, the mouse was perfused with fixative through the circulatory system to obtain the best possible preservation of the spinal cord for immunohistochemistry. Briefly, 1.5 g/kg urethane was administered via intraperitoneal injection. After the mouse became unresponsive, a 5-6 cm lateral incision was made through the abdominal wall just beneath the rib cage and the diaphragm was broken. The pleural cavity was fully exposed and a needle connected with a perfusion pump was directly inserted into the protrusion of left ventricle. Then, the mouse right atrium was opened using iris scissors to allow the blood to flow out of the body. Mice were first perfused with 0.9% saline and switched to 4% paraformaldehyde (PFA) when the fluid was running clear. After the perfusion fixation, the spinal cord was separated and immersed in 4% PFA in a 15 ml tube for another 24 hours at 4° C. and then transferred into 30% sucrose/PBS solution at 4° C. until the tissue sank to the bottom. The fixed spinal cord was embedded in OCT on dry ice and sliced into 30 μm sections using a Leica cryostat. The slices were kept in PBS at 4° C. before immunohistochemistry. The slices were then blocked with 0.1% Triton X-100 and 5% BSA in PBS for 1 hour. Then, the slices were incubated with primary antibody at 1:100 in 3% BSA in PBS overnight at 4° C. On the second day, slices were washed with PBS 3 times and incubated with secondary antibody with Alxe-488 or Alxe-568 at 1:200 for 2 hours at room temperature. After washing with PBS again, slices were stained with DAPI at 1:500 for 5 minutes. Then the samples were washed with PBS 3 times and mounted onto the glass slices. Sections were then visualized and imaged using a confocal microscope. The following primary antibodies were used in the study: anti-phosphoTDP-43 (409/410) (Cosmo, CAC-TIP-PTD-M01), anti-TDP-43 (Proteintech, 10782-2-AP), anti-HSP70 (Abcam, ab2787) and anti-TIA1 (Abcam, ab263945).

Peptide preparation and administration in mice: For peptide intravenous (iv) injection in the mouse model, the peptides were dissolved in saline and prepared freshly on the day of experiment at a stock concentration of 4 mg/mL. 20 mg/kg (5 mg/kg of each of four peptides) peptides were each administered once per day through intravenous injection via the tail vein. The first injection took place 30 minutes before the surgical procedure and the following injections took place every 24 hours until the mice were sacrificed.

Sciatic nerve axotomy procedure in mice: 6-8 week old adult male C57BL/6 mice weighing 20-25 g were employed for the sciatic nerve axotomy procedure. Mice were anaesthetized with 5% isoflurane in 70% N₂O/30% O₂ mixed gas in the induction chamber and maintained with 2% isoflurane in 70% N₂O/30% O₂ using a nose cone. Once anesthetized, mice were placed on a thermal pad and body temperature was maintained at 37.5° C. Ophthalmic ointment was applied to both eyes. To control the pain in the procedure, 5 mg/kg ketoprofen and 0.05 mg/kg buprenorphine were given subcutaneously prior to the surgery. The fur was shaved between the cranial edge of the wing of the ileum and the last rib on the left side and the skin was prepared for surgery with alternating scrub of iodine solution and 70% ethanol (three repeats). A clean surgical drape was placed over the body with an appropriate operative field. Next, a 0.5 cm skin incision was made parallel to the femur. The sciatic nerve was exposed after opening the fascial plane between the gluteus maximus and the anterior head of the biceps femoris. Then the sciatic nerve was gently freed from the surrounding connective tissue using iridectomy scissors and transected in the middle. After the procedure, the muscle, subcutaneous tissue and the skin were closed with 4-0 vicryl suture layer by layer. Gas anesthesia was turned off and mice were placed into a recovery cage and monitored for another 2 hours for adequate recovery. The mice were provided with supportive care after the surgery and body weight was monitored for before and after surgery. At day 1 and day 2 after surgery, ketoprofen (5 mg/kg) and buprenorphine (0.05 mg/kg) were given subcutaneously to control the pain. The mice were sacrificed at various times (days 1 to 7) after surgery.

### Preliminary Example. Investigating the Functions and Relationships of CK-1δ and TDP-43

To confirm the function of CK-1δ as the general mechanism for TDP-43 phosphorylation, either the empty vector, pCS2-Myc-CK1δ or pcDNA3.1-FLAG-CK1δ1-317 was overexpressed in HEK293 cells. Endogenous TDP-43 was highly phosphorylated by active CK-1δ (lacking its regulatory domain) in a time-dependent manner (FIGS. 9A and 9B). It was further confirmed that the interactions between TDP-43 and CK-1δ were not stable, since there was no clear binding in a co-immunoprecipitation assay (FIG. 10).

Studies were also conducted to investigate whether CK-1δ-caused TDP-43 phosphorylation is related to two common pathological features, TDP-43 aggregation and cytoplasmic redistribution. The insoluble fraction of total protein was separated at 48 hours after the HEK293 cells were transfected with either empty vector or pcDNA3.1-FLAG-CK1δ 1-317. Both phosphorylated and total TDP-43 abundantly accumulated in the insoluble fraction with pcDNA3.1-FLAG-CK1δ 1-317 overexpression (FIG. 1A).

Further studies were conducted to explore whether phosphorylation of TDP-43 was responsible for its cytoplasmic redistribution. The proteins both in nucleus and cytoplasm from either empty vector or pcDNA3.1-FLAG-CK1δ 1-317 overexpression were quantitively measured by Western immunoblotting. The phosphorylated TDP-43 was concentrated in the nuclear fraction and endogenous TDP-43 did not show cytoplasmic redistribution following the pcDNA3.1-FLAG-CK1δ 1-317 overexpression (FIG. 1B). In immunocytochemistry experiments, 75.3% of the pcDNA3.1-FLAG-CK1δ 1-317 expressing cells were positive for staining of phosphorylated TDP-43. In addition, phosphorylated TDP-43 and CK1δ (1-317) were colocalized in aggregates in the nucleus (FIG. 1C) providing direct evidence that active CK1δ (CK1δ 1-317) can decrease the solubility of endogenous TDP-43, but does not cause cytoplasmic redistribution. These results suggested TDP-43 phosphorylation is related to decreased solubility, and TDP-43 cytoplasmic redistribution under the pathological conditions is an event that is independent from TDP-43 phosphorylation.

Disruption of TDP-43 Nuclear-Cytoplasmic Transportation was Associated with Decreased Solubility

Further studies were performed to disrupt TDP-43's normal shuttling from nucleus to cytoplasm and vice versa by mutating the sequences in the nuclear localization signal and nuclear export signal, in order to investigate how the cellular location of TDP-43 was correlated with phosphorylation and aggregation (FIG. 2A). Mutations in the nuclear localization signal (NLS) or nuclear export signal (NES) have been reported to disrupt TDP-43 nuclear-cytoplasmic shuttling and sequester the protein in either cytoplasm or nucleus respectively, despite the well-known findings that TDP-43 has a predominant nuclear localization under normal circumstances (Winton et al., 2008). Truncated CK1δ (1-317) was then co-expressed with either TDP-43 wildtype, ΔNLS or ΔNES in HEK293 cells and the proteins in the soluble or insoluble fraction of cell lysates were analyzed using immunoblotting. TDP-43 ΔNES self-aggregated in the cells whereas TDP-43 wildtype and TDP-43 ΔNLS maintained good solubility. When co-expressed with truncated CK1δ (1-317), the solubility of TDP-43 wildtype, ΔNLS and ΔNES were all decreased, showing as increased protein density in the insoluble fraction. Interestingly, both TDP-43 ΔNLS and ΔNES became more insoluble than wildtype after phosphorylation and TDP-43 ΔNES changed the most among the three (FIG. 2B). These results demonstrate that neither keeping the TDP-43 in the nucleus nor in the cytoplasm could protect them from aggregation, suggesting that decreased TDP-43 trafficking may occur in disease progression and promote TDP-43 aggregate formation.

Wild Type TDP-43 and Phosphorylated TDP-43 Share the Same Group of Protein Partners but with Different Binding Affinity

The protein-protein interaction networks of wildtype TDP-43 and phosphorylated TDP-43 were also explored. HEK293 cells were transfected with either empty vector or pcDNA3.1-FLAG-CK1δ 1-317 for 48 hours and proteins were pulled down using a TDP-43 antibody or phosphoTDP-43 antibody. The protein partners were analyzed by mass spectrometry. The control was represented as immobile beads only and the corresponding pulled-down proteins were dimethyl labelled light. Proteins were pulled down using either TDP-43 antibody or phosphoTDP-43 antibody dimethyl labelled medium or heavy respectively. Wildtype TDP-43 and phosphorylated TDP-43 shared most of the same protein partners but with different binding affinities. The binding ratios indicate that phosphorylated TDP-43 lost some of its binding with various proteins suggesting a partial loss of function. Interestingly, phosphorylated TDP-43 obtained two new binding partners (Zinc finger protein 320 and Protein ZNF767) which were not protein partners for wildtype TDP-43.

### Experimental Example 1. Development of Example Peptides a, B, C, and D

To identify the binding region for TDP-43, a peptide array containing 12 mers with a 2 amino acid shift to cover the full sequence of CK-1δ was employed. Four potential binding regions were identified when probed with TDP-43 protein on the CK-1δ membrane (FIG. 3A). This enabled the inventors hereof to derive four candidate peptides (example peptide A, SEQ ID NO:1; example peptide B, SEQ ID NO:2; example peptide C, SEQ ID NO:3; example peptide D, SEQ ID NO:4) which could interfere with CK-1δ and TDP-43 interactions. The effects of different combinations of the four peptide candidates on blocking the interactions between TDP-43 and CK-1δ were tested at a concentration of 20 μM (FIG. 3B). The efficiency of different doses (0, 5, 10, 15, 20, 25 μM) and combinations of peptides on TDP-43 phosphorylation blockade were also tested (FIG. 10). A mixture of the four example peptides in equal parts (A+B+C+D) was found to block TDP-43 phosphorylation at concentrations as low as 5 μM for each peptide.

The example peptides' specificity was investigated by examining another known CK1δ substrate, the p53 protein, to assess the potential for off target effects. CK1δ has been reported to phosphorylate p53 at multiple sites and to play a major role in the cellular response to DNA damage or other cellular stresses (MacLaine et al., 2008). In the present studies, p53 was phosphorylated by overexpression of pcDNA3.1-FLAG-CK1δ1-317 and the peptide combination (A+B+C+D) had no effect on p53 phosphorylation (FIG. 3C). These results indicate that the peptides herein candidates specifically blocked the interaction between CK1δ and TDP-43 while sparing other CK1δ substrates, namely, p53.

The effects of this peptide mixture (A+B+C+D) on CK1δ overexpression-induced TDP-43 aggregation were also evaluated. TDP-43 in the insoluble fraction was increased with pcDNA3.1-FLAG-CK1δ1-317 overexpression and the level of insoluble TDP-43 was reduced with treatment by the peptides herein. The level of phosphorylated TDP-43 was reduced with peptide mixture treatment in both the soluble fraction and the insoluble fraction (FIG. 3D). Thus, peptides as described herein can efficiently block TDP-43 phosphorylation and partially reduce TDP-43 aggregation induced by CK1δ overexpression.

### Experimental Example 2. Development of Example Peptide ppTDP

A further example peptide was prepared using a blocking sequence derived from the phosphorylation sites of TDP-43.

In vitro studies have reported 29 TDP-43 phosphorylation sites potentially targeted by CK-1δ (Kametani et al., 2009) (FIG. 4A). To assess how each phosphorylation site contributed to TDP-43 aggregation, each amino acid of these 29 residues was substituted to Alanine to prevent potential phosphorylation. Then, each vector was co-expressed with pcDNA3.1-FLAG-CK1δ1-317. Surprisingly, seven of these substitutions increased TDP-43 aggregation and only two of them showed less insolubility than wildtype (FIG. 4B). The inventors hereof further tested the expression pattern of these expressing vectors only. The T88A, S91A, S92A, T116A, S254A, S305A and S369A mutations led to self-aggregation in HEK293 cells suggesting that these aggregations were not caused by CK1δ phosphorylation and might be caused by structural changes. Finally, studies were conducted to reconfirm the solubility of T25A, S403A and two previous reported phosphorylation sites (S409A and S410A) together with pcDNA3.1-FLAG-CK1δ1-317. T25A, S403A and the combination of S409A and S410A dramatically restored solubility when compared with wildtype TDP-43 (FIG. 4C).

Accordingly, another example peptide according to the present description was prepared that covered the T25 site at the N-terminal and S403 site at the C-terminal of TDP-43, with a linker sequence between the two blocking sequences and a TAT sequence to facilitate cell entry (referred to herein as the “ppTDP” peptide; SEQ ID NO:5) (FIG. 4D). To investigate whether this peptide could also block the interaction between TDP-43 and CK1δ, pcDNA3.1-FLAG-CK1δ1-317 was overexpressed in HEK293 cells and the cells were treated with the ppTDP peptide at 20 μM or 40 μM every 6 hours for a total of 48 hours. As shown in FIG. 4E, the ppTDP peptide at 20 μM efficiently blocked TDP-43 phosphorylation in both the soluble fraction and insoluble fraction. However, the ppTDP peptide at 40 μM did not block TDP-43 aggregation induced by CK1δ more effectively.

### Experimental Example 3. Investigation of Example Peptides in Neuronal Cellular Models

Ethacrynic acid (EA) has been reported to induce TDP-43 phosphorylation and aggregation in SH-SY5Y cells (Iguchi et al., 2012). The present inventors first investigated whether phosphoTDP-43 redistribution to the cytoplasm is a feature in this model. The SH-SY5Y cells were treated with 100 μM ethacrynic acid for 24 hours and fixed for immunocytochemistry. The visualized phosphoTDP-43 was mainly located in the cytoplasm showing a nuclear clearance pattern which is similar to that observed in pathological findings in neurodegenerative diseases such as ALS and FTLD (FIG. 5A). Next, endogenous CK-1δ was knocked down and the SH-SY5Y cells were treated with 100 μM ethacrynic acid for 24 hours. Both TDP-43 and phosphoTDP-43 were not seen in the insoluble fraction with the treatment of ethacrynic acid, suggesting that CK-1δ is necessary for ethacrynic acid induced TDP-43 phosphorylation and aggregation in SH-SY5Y cells (FIG. 5B).

To assess the effects of exemplary peptides hereof on ethacrynic acid induced TDP-43 aggregation, SH-SY5Y cells or HEK293 cells were pre-treated with a mixture of peptides A+B+C+D for 1 hour and ethacrynic acid was added for another 24 hours to induce TDP-43 aggregation. Interestingly, the ethacrynic acid induced TDP-43 aggregation was only seen in SH-SY5Y cells but not HEK293 cells, suggesting that this toxicity-induced decreased protein solubility is a unique feature in neuronal cells (FIG. 5C). With the treatment with peptide mixture at 20 μM, both TDP-43 and phosphoTDP-43 in the insoluble fraction were totally absent. Similar results were obtained from primary cultured cortical neurons treated with 20 μM ethacrynic acid. The peptide mixture (at 5 μM concentration for each peptide) efficiently blocked TDP-43 aggregation (FIG. 5D).

Peptides according to the present description were also tested in SH-SY5Y cells challenged with sodium arsenite, a known chemical that induces oxidative stress. With the treatment of 1 mM sodium arsenite for 3 hours, TDP-43 and phosphoTDP-43 were heavily accumulated in the insoluble fraction of SH-SY5Y cells. Consistent results on reducing insoluble TDP-43 and phosphoTDP-43 were obtained from cells treated with either the peptide mixture (A+B+C+D) or peptide D. Notably, peptide mixture (A+B+C+D) or peptide D treatment also showed less PARP cleavage caused by sodium arsenite, indicating apoptosis was reduced or prevented (FIG. 5E). Treatment with Peptide D also reduced the number of stress granules (SGs) induced by sodium arsenite (FIG. 5F).

These results indicate that blocking the interaction of CK-1δ and TDP-43 by use of the present peptides can efficiently reduce TDP-43 phosphorylation and aggregation in different neuronal models.

### Experimental Example 4. Use of Example Peptides to Partially Rescue Cell Death in Relevant Cellular Models

The cell viability of SH-SY5Y cells under each condition were determined using an MTT assay. Compared with the control group, ethacrynic acid treatment caused 69.97% cell death of SH-SY5Y cells. Remarkably, pre-treatment with example peptides as described herein (A+B+C+D) effectively rescued 27.44% of cells from ethacrynic acid induced cell death (FIG. 5G). In primary cultured cortical neurons, cell viability was assessed by measuring LDH release into the culture medium after ethacrynic acid treatment. The peptide mixture at 5 μM (1.25 μM for each peptide) reduced the number of dead neurons by about half (from 18.82% to 9.81%) (FIG. 5H).

### Experimental Example 5. Use of Example Peptides to Reduce TDP-43 Phosphorylation and Aggregation In Vivo

To explore the effects of example peptides according to the present description in vivo, a retrograde degeneration mouse model was employed. This was done by crushing the sciatic nerve, which has been shown to lead to TDP-43 proteinopathy in the ventral horn motor neurons of the spinal cord (Moisse et al., 2009). C57BL/6 mice were subjected to left sciatic nerve axotomy and the spinal cord tissues were collected on each day from day 0 to day 7 after surgery. The subcellular localization of TDP-43 and phosphoTDP-43 were assessed in this model. In the side ipsilateral to the sciatic nerve axotomy, both TDP-43 and phosphoTDP-43 were found in the cytoplasm of motor neurons on days 3, 4 and 5 postsurgery in the ventral horn of the lumbar spinal cord. On the side of the spinal cord contralateral to the surgery, TDP-43 was concentrated in the nucleus as it is in normal animals (FIG. 6A). This phenotype is similar to that observed in pathological samples in neurodegenerative diseases like ALS, although here it is a transient phenomenon. To determine the solubility of TDP-43 and phosphoTDP-43 in this model, the proteins were extracted from ipsilateral lumbar spinal cord for immunoblotting on each day from day 0 to day 7 after surgery. As shown in FIG. 6B, the protein levels of TDP-43 and phosphoTDP-43 were increased in the insoluble fraction on day 3, 4 and 5 in parallel with a decrease in the soluble fraction and these changes returned to baseline at day 7.

To test the effects of example peptides hereof in the spinal cord response to axotomy, peptides (mixture of A+B+C+D) were injected at 20 mg/kg into C57BL/6 mice tail vein once a day from day 0 to day 7 and protein levels were compared with those from a scrambled peptide injection group. With the peptide mixture injection, TDP-43 was less abundant in the insoluble fraction and phosphoTDP-43 was only slightly visible on day 3 (FIG. 6B), demonstrating the effectiveness of the peptides in blocking TDP-43 phosphorylation in vivo. To quantitatively compare TDP-43 levels in the peptide mixture group versus scrambled peptide group, TDP-43 expression was analyzed on days 3, 4 and 5 post-axotomy in two groups. TDP-43 levels in the peptide mixture group in the insoluble fraction were significantly reduced by 50.17%, 29.03% and 14.85% on day 3, 4 and 5 when compared with that of the scrambled peptide group (FIG. 6C). Together, these results demonstrated that the peptide mixture derived from CK-1δ efficiently blocked TDP-43 phosphorylation and partially reduced TDP-43 aggregation in vivo. However, the cellular localization of TDP-43 in days 3, 4 and 5 after sciatic nerve axotomy with peptide mixture treatment was still concentrated in the cytoplasm without nuclear rescue (FIG. 6D) suggesting that TDP-43 phosphorylation is not the cause of TDP-43 nuclear clearance in this model.

### Experimental Example 6. Treatment with Example Peptides does not Affect HSP70 Activity after Sciatic Nerve Axotomy

The accumulation of misfolded proteins in the CNS is a major feature of many neurodegenerative diseases and the heat shock protein (HSP) family of proteins are involved in each step of proteostasis that facilitates protein folding and degradation by ubiquitin-proteasome or autophagy pathways. Heat shock proteins have been implicated in TDP-43 clearance in transgenic mouse models and human ALS tissue (Chen et al., 2016). In response to stress, it has been demonstrated that the expression of heat shock protein 70 is upregulated and that the nuclear translocation is important for suppression of glutamate excitotoxicity-induced apoptosis (Song et al., 2016). In the present studies, the levels of TDP-43 and phosphoTDP-43 were increased in the insoluble fraction after sciatic nerve axotomy. Further tests were then conducted to assess whether the protein level of heat shock protein 70 (HSP70) was affected by sciatic nerve axotomy since HSP family members were found to be upregulated during stress exposure, perhaps to prevent additional conformational changes or self-aggregation of misfolded proteins. However, in this model, HSP70 levels were not regulated after sciatic nerve axotomy within 7 days in both control group and peptide group (FIG. 7A). Interestingly, HSP70 did show a nuclear translocation on days 1, 2 and 3 after sciatic nerve axotomy which suggested HSP70 activation and the initiation of a heat shock response in this system. Comparing this phenomenon in control and peptide mixture-treated groups revealed that the peptide mixture that blocked TDP-43 phosphorylation did not alter the nuclear translocation of HSP70 (FIG. 7B), suggesting that that TDP-43 phosphorylation was not involved in the initial heat shock response after sciatic nerve axotomy.

### Experimental Example 7. Treatment with Example Peptides Reduces Stress Granule Formation Induced by Sciatic Nerve Axotomy

Stress granules (SGs) are formed in the cytoplasm in response to cellular stress. Previous studies have demonstrated that post-translational modification of RNA binding proteins strongly contributes to the regulation of SGs (Lee et al., 2012; Hofweber and Dormann, 2019). Pathological proteins containing low complexity domains in which the glycine and proline are overrepresented and with hydrophobic side groups are recruited into stress granules under pathological conditions and the maturation of stress granules increase the probability of these proteins forming potential fibrillar structures (Dobra et al., 2018). Therefore, the effects of example peptides on stress granule formation after sciatic nerve axotomy were investigated by assessing the stress granule marker TIA-1.

A mixture of example peptides or scrambled control peptides were injected at 20 mg/kg into C57BL/6 mice tail vein once a day from day 0 to day 7 after sciatic nerve axotomy. Immunohistochemical staining of TIA-1 in the control group was concentrated within cytoplasmic foci in the ipsilateral lumbar spinal cord slices which indicated stress granules had formed after acute peripheral nerve injury. These stress granules existed on day 1, 2 and 3 and disassembled gradually after. Surprisingly, these cytoplasmic foci were not observed in the peptide mixture group (FIG. 8A), suggesting that TDP-43 phosphorylation regulates sciatic nerve axotomy induced stress granule assembly.

The protein level of TIA-1 was also quantitatively analyzed by western immunoblotting. TIA-1 was dramatically increased in the insoluble fraction on the day 1, 2 and 3 after sciatic nerve axotomy in the control group. However, TIA-1 in the insoluble fraction was not abundantly observed in the peptide mixture injection group (FIG. 8B). Comparison of TIA-1 levels in the insoluble fraction in two groups on the same blots further confirmed the effects of peptide mixture on reducing stress granule assembly on days 1, 2 and 3 after sciatic nerve axotomy (FIG. 8C). These results suggest that TDP-43 phosphorylation is crucial both for stress granule assembly and protein aggregate formation after axotomy, and that inhibiting such phosphorylation may have ameliorative effects on both processes.

### Experimental Example 8. Reduction of TDP-43 and phosphoTDP43 Aggregates in HL-1 Cells with Sodium Arsenite Treatment in Muscle Cells

In order to evaluate the function of example peptide D on alleviation of TDP-43 proteinopathies in cardiomyocytes, 0.05 mM of sodium arsenite was employed to treat HL-1 cells for 16 hours to induce TDP-43 and phosphoTDP43 aggregates. As shown in FIG. 12, sodium arsenite treatment reduced TDP-43 solubility, seen as decreased expression in the soluble fraction and increased expression in the insoluble fraction, as well as an increase of insoluble phosphorylated TDP-43. 20 μM of peptide D treatment in HL-1 cells under the same treatment conditions reduced insoluble aggregates of TDP-43 and phosphoTDP-43. In addition, PARP cleavage, a protease marker of cell toxicity, was evaluated in peptide D treated vs untreated cells, and peptide D treatment reduced PARP cleavage product. These results suggest that example peptide D reduces TDP-43 and phosphoTDP-43 aggregation in HL-1 cells and partially inhibits the activation of the PARP cell death protease.

### Experimental Example 9. Dose Dependent Reduction of TDP-43 and phosphoTDP-43 Aggregates and Prevention of Neuronal Degeneration In Vivo

As previously discussed, the present inventors have found that sciatic nerve axotomy in mice can induce an increase of insoluble TDP-43 and phosphoTDP-43 in the ventral horn of the spinal cord on day 3, 4, 5 post-surgery that recovers after 7 days. This mouse model was further optimized by adding one dose of lipopolysaccharide (LPS) injection (5 mg/kg, i.p.) when performing surgery. LPS injection aggravated the observed TDP-43 proteinopathy as assessed at day 3 post-surgery. Treatment with example peptide D reduced TDP-43 and phosphoTDP-43 aggregation in a dose dependent manner (FIG. 13).

Neurodegeneration in the lumbar spinal cord of mice 7 days after administration of LPS plus surgery was then evaluated using Fluoro Jade C staining. Animals were divided into two peptide D treatment groups. In one group, peptide D administration started on the day of surgery, and in the other group, peptide D treatment started at 3 days post-surgery when the TDP-43 proteinopathies were at their maximum. The results indicate that both treatment groups had less neurodegeneration in the ventral horn of spinal cord than was observed in the control group (FIG. 14).

These results indicate that administration of example peptide D can prevent neurodegeneration and neuroinflammation when administered at the time of surgery, and can also reduce neurodegeneration and neuroinflammation when administered at the time in the process when TDP pathology is at its maximum.

### Experimental Example 10. Reduction of Microglia Activation and Prevention of Motor Neuron Death in Ventral Horn of Spinal Cord of Mice with LPS Plus Sciatic Nerve Axotomy

Although neurodegeneration was detected in spinal cord of mice treated with a high dose of LPS (5 mg/kg, i.p.) plus sciatic nerve axotomy, motor neuron death was not observed in this model. To replicate this prominent feature of ALS, a low dose of LPS (1 mg/kg, i.p.) injection but with longer duration of administration (once daily for 7 days) was employed instead of one administration of a high dose of LPS (5 mg/kg, i.p.).

Since LPS causes systematic inflammation, microglia activation in ventral horn of spinal cord was first detected by staining with an Iba-1 antibody. As shown in FIG. 15, example peptide D treatment reduced general microglia activation caused by LPS injection in lumbar spinal cord. In this setting, motor neuron loss in the lower limb motoneuron group in L5/6 of spinal cord was observed, as confirmed by staining with ChAT and NeuN antibodies. Mice with peptide D treatment showed less motor neuron death on day 7 post-surgery, which suggests that peptide D can protect motor neurons from the combined insult of LPS treatment plus sciatic nerve axotomy (FIG. 16). TDP-43 and phosphoTDP-43 insolubility in lumbar spinal cord was also confirmed by western blotting. Peptide D treatment reduced TDP-43 aggregation when compared with control mice (FIG. 17).

Altogether, these results indicate that peptide D treatment can reduce microglia activation induced by LPS and prevent motor neuron death in ventral horn of spinal cord of mice treated with LPS plus sciatic nerve axotomy, and may be expected to show similar results in other animals having TDP-43 proteinopathies.

### Clauses

Clause 1. A peptide comprising: (a) a cell membrane penetration (CMP) sequence; and (b) one or more TDP-43 phosphorylation-blocking (PB) sequences.

Clause 2. The peptide according to clause 1, wherein the PB sequence comprises an amino acid sequence having at least about 90% identity with the amino acid sequence of SEQ ID NO: 10.

Clause 3. The peptide according to clause 1, wherein the PB sequence comprises an amino acid sequence having at least about 95% identity with the amino acid sequence of SEQ ID NO:10.

Clause 4. The peptide according to clause 1, wherein the PB sequence comprises the amino acid sequence of SEQ ID NO:10.

Clause 5. The peptide according to clause 1, wherein the PB sequence comprises an amino acid sequence having at least about 90% identity with the amino acid sequence of SEQ ID NO: 11.

Clause 6. The peptide according to clause 1, wherein the PB sequence comprises an amino acid sequence having at least about 95% identity with the amino acid sequence of SEQ ID NO:11.

Clause 7. The peptide according to clause 1, wherein the PB sequence comprises the amino acid sequence of SEQ ID NO:11.

Clause 8. The peptide according to any one of clauses 1 to 7, wherein the CMP sequence comprises an amino acid sequence derived from the HIV-1 Tat domain.

Clause 9. The peptide according to clause 8, wherein the peptide comprises an amino acid sequence having at least about 90% identity with the amino acid sequence of one of SEQ ID NO:1, SEQ ID NO:2, SEQ ID NO:3, SEQ ID NO:4, or SEQ ID NO:5.

Clause 10. The peptide according to clause 8, wherein the peptide comprises an amino acid sequence having at least about 95% identity with the amino acid sequence of one of SEQ ID NO:1, SEQ ID NO:2, SEQ ID NO:3, SEQ ID NO:4, or SEQ ID NO:5.

Clause 11. The peptide according to clause 8, wherein the peptide comprises the amino acid sequence of one of SEQ ID NO:1, SEQ ID NO:2, SEQ ID NO:3, SEQ ID NO:4, or SEQ ID NO:5.

Clause 12. A peptide, or a pharmaceutically acceptable salt or derivative thereof, comprising an amino acid sequence having at least about 90% identity with the amino acid sequence of SEQ ID NO:4.

Clause 13. A peptide, or a pharmaceutically acceptable salt or derivative thereof, comprising an amino acid sequence having at least about 95% identity with the amino acid sequence of SEQ ID NO:4.

Clause 14. A peptide, or a pharmaceutically acceptable salt or derivative thereof, comprising the amino acid sequence of SEQ ID NO:4.

Clause 15. A peptide comprising: (a) a cell membrane penetration (CMP) sequence; (b) a first TDP-43 phosphorylation-blocking (PB) sequence; (c) a second TDP-43 phosphorylation-blocking (PB) sequence; and (d) optionally, a linker sequence.

Clause 16. A peptide, or a pharmaceutically acceptable salt or derivative thereof, comprising an amino acid sequence having at least about 90% identity with the amino acid sequence of SEQ ID NO:5.

Clause 17. A peptide, or a pharmaceutically acceptable salt or derivative thereof, comprising an amino acid sequence having at least about 95% identity with the amino acid sequence of SEQ ID NO: 5.

Clause 18. A peptide, or a pharmaceutically acceptable salt or derivative thereof, comprising the amino acid sequence of SEQ ID NO:5.

Clause 19. The peptide according to any one of clauses 1 to 18, wherein the peptide is prepared by chemical synthesis.

Clause 20. A peptide according to any one of clauses 1 to 19 for use in reducing phosphorylation of TDP-43.

Clause 21. A peptide according to any one of clauses 1 to 20 for use in reducing aggregation of TDP-43.

Clause 22. A peptide according to any one of clauses 1 to 21 for use in reducing the formation of stress granules.

Clause 23. A peptide according to any one of clauses 1 to 22 for use in reducing or interfering with an interaction between TDP-43 and CK1δ.

Clause 24. A method of reducing the phosphorylation of TDP-43 in a cell, the method comprising: delivering an effective amount of a peptide to the cell, wherein the peptide comprises: (a) a cell membrane penetration (CMP) sequence; and (b) a TDP-43 phosphorylation-blocking (PB) sequence.

Clause 25. The method according to clause 24, wherein the aggregation of TDP-43 in the cell is prevented or reduced.

Clause 26. The method according to clause 24, wherein the formation of stress granules in the cell is prevented or reduced.

Clause 27. The method according to any one of clauses 24 to 26, wherein the delivery of the peptide to the cell is effected in vitro.

Clause 28. The method according to any one of clauses 24 to 26, wherein the delivery of the peptide to the cell is effected ex vivo.

Clause 29. The method according to any one of clauses 24 to 26, wherein the delivery of the peptide to the cell is effected in vivo.

Clause 30. The method according to any one of clauses 24 to 29, wherein the PB sequence comprises an amino acid sequence having at least about 90% identity with the amino acid sequence of SEQ ID NO:10.

Clause 31. The method according to any one of clauses 24 to 29, wherein the PB sequence comprises an amino acid sequence having at least about 95% identity with the amino acid sequence of SEQ ID NO:10.

Clause 32. The method according to any one of clauses 24 to 29, wherein the PB sequence comprises the amino acid sequence of SEQ ID NO:10.

Clause 33. The method according to any one of clauses 24 to 29, wherein the PB sequence comprises an amino acid sequence having at least about 90% identity with the amino acid sequence of SEQ ID NO:11.

Clause 34. The method according to any one of clauses 24 to 29, wherein the PB sequence comprises an amino acid sequence having at least about 95% identity with the amino acid sequence of SEQ ID NO:11.

Clause 35. The method according to any one of clauses 24 to 29, wherein the PB sequence comprises the amino acid sequence of SEQ ID NO:11.

Clause 36. The method according to any one of clauses 24 to 35, wherein the CMP sequence comprises an amino acid sequence derived from the HIV-1 Tat domain.

Clause 37. The method according to clause 36, wherein the peptide comprises an amino acid sequence having at least about 90% identity with the amino acid sequence of SEQ ID NO:4.

Clause 38. The method according to clause 36, wherein the peptide comprises an amino acid sequence having at least about 95% identity with the amino acid sequence of SEQ ID NO:4.

Clause 39. The method according to clause 36, wherein the peptide comprises the amino acid sequence of SEQ ID NO:4.

Clause 40. The method according to clause 36, wherein the peptide comprises an amino acid sequence having at least about 90% identity with the amino acid sequence of SEQ ID NO:5.

Clause 41. The method according to clause 36, wherein the peptide comprises an amino acid sequence having at least about 95% identity with the amino acid sequence of SEQ ID NO:5.

Clause 42. The method according to clause 36, wherein the peptide comprises the amino acid sequence of SEQ ID NO:5.

Clause 43. A method of treating a disease or pathological condition, the method comprising administering an effective amount of a peptide to a subject in need thereof; wherein the peptide comprises: (a) a cell membrane penetration (CMP) sequence; and (b) one or more TDP-43 phosphorylation-blocking (PB) sequences.

Clause 44. The method according to clause 43, wherein the subject is a human.

Clause 45. The method according to clause 43 or clause 44, wherein the disease or pathological condition is associated with phosphorylation and/or aggregation of TDP-43.

Clause 46. The method according to any one of clauses 43 to 45, wherein the disease or pathological condition is associated with the formation or presence of stress granules.

Clause 47. The method according to any one of clauses 43 to 46, wherein the disease or pathological condition is a neurodegenerative disease.

Clause 48. The method according to clause 47, wherein the neurodegenerative disease is amyotrophic lateral sclerosis (ALS), frontotemporal dementia (FTD), or limbic-predominant age-related TDP-43 encephalopathy (LATE).

Clause 49. The method according to clause 47 or clause 48, wherein the neurodegenerative disease is ALS.

Clause 50. The method according to clause 47 or clause 48, wherein the neurodegenerative disease is FTD.

Clause 51. The method according to clause 47 or clause 48, wherein the neurodegenerative disease is LATE.

Clause 52. The method according to any one of clauses 43 to 51, wherein the administering of the effective amount of the peptide is by systemic administration.

Clause 53. The method according to any one of clauses 43 to 52, wherein the PB sequence comprises an amino acid sequence having at least about 90% identity with the amino acid sequence of SEQ ID NO:10.

Clause 54. The method according to any one of clauses 43 to 52, wherein the PB sequence comprises an amino acid sequence having at least about 95% identity with the amino acid sequence of SEQ ID NO:10.

Clause 55. The method according to any one of clauses 43 to 52, wherein the PB sequence comprises the amino acid sequence of SEQ ID NO:10.

Clause 56. The method according to any one of clauses 43 to 52, wherein the PB sequence comprises an amino acid sequence having at least about 90% identity with the amino acid sequence of SEQ ID NO:11.

Clause 57. The method according to any one of clauses 43 to 52, wherein the PB sequence comprises an amino acid sequence having at least about 95% identity with the amino acid sequence of SEQ ID NO:11.

Clause 58. The method according to any one of clauses 43 to 52, wherein the PB sequence comprises the amino acid sequence of SEQ ID NO:11.

Clause 59. The method according to any one of clauses 43 to 52, wherein the peptide comprises an amino acid sequence having at least about 90% identity with the amino acid sequence of SEQ ID NO:4.

Clause 60. The method according to any one of clauses 43 to 52, wherein the peptide comprises an amino acid sequence having at least about 95% identity with the amino acid sequence of SEQ ID NO:4.

Clause 61. The method according to any one of clauses 43 to 52, wherein the peptide comprises the amino acid sequence of SEQ ID NO:4.

Clause 62. The method according to any one of clauses 43 to 52, wherein the peptide comprises an amino acid sequence having at least about 90% identity with the amino acid sequence of SEQ ID NO:5.

Clause 63. The method according to any one of clauses 43 to 52, wherein the peptide comprises an amino acid sequence having at least about 95% identity with the amino acid sequence of SEQ ID NO:5.

Clause 64. The method according to any one of clauses 43 to 52, wherein the peptide comprises the amino acid sequence of SEQ ID NO:5.

Clause 65. A vector encoding a peptide according to any one of clauses 1 to 23.

Clause 66. A polynucleotide encoding a peptide according to any one of clauses 1 to 23.

Clause 67. A pharmaceutical composition comprising: an effective amount of a peptide according to any one of clauses 1 to 23, or a pharmaceutically acceptable salt or derivative thereof; and at least one pharmaceutically acceptable excipient or adjuvant.

### REFERENCES

Arai T, Hasegawa M, Akiyama H, et al. TDP-43 is a component of ubiquitin-positive tau-negative inclusions in frontotemporal lobar degeneration and amyotrophic lateral sclerosis. Biochem Biophys Res Commun. 2006; 351(3):602-611. doi:10.1016/j.bbrc.2006.10.093
Baskaran P, Shaw C, Guthrie S. TDP-43 causes neurotoxicity and cytoskeletal dysfunction in primary cortical neurons. PLoS One. 2018; 13(5):e0196528. doi:10.1371/joumal.pone.0196528
Becker L A, Huang B, Bieri G, et al. Therapeutic reduction of ataxin-2 extends lifespan and reduces pathology in TDP-43 mice. Nature. 2017; 544(7650):367-371. doi:10.1038/nature22038
Bentmann E, Neumann M, Tahirovic S, Rodde R, Dormann D, Haass C. Requirements for stress granule recruitment of fused in sarcoma (FUS) and TAR DNA-binding protein of 43 kDa (TDP-43). J Biol Chem. 2012; 287(27):23079-23094. doi:10.1074/jbc.M111.328757
Carlomagno Y, Zhang Y, Davis M, et al. Casein kinase II induced polymerization of soluble TDP-43 into filaments is inhibited by heat shock proteins. PLoS One. 2014; 9(3):e90452. doi:10.1371/joumal.pone.0090452
Chen H J, Mitchell J C, Novoselov S, et al. The heat shock response plays an important role in TDP-43 clearance: evidence for dysfunction in amyotrophic lateral sclerosis. Brain. 2016; 139(Pt 5):1417-1432. doi:10.1093/brain/aww028
Chen Y, Cohen T J. Aggregation of the nucleic acid-binding protein TDP-43 occurs via distinct routes that are coordinated with stress granule formation. J Biol Chem. 2019; 294(10):3696-3706. doi:10.1074/jbc.RA118.006351
Chiang H H, Andersen P M, Tysnes O B, Gredal O, Christensen P B, Graff C. Novel TARDBP mutations in Nordic ALS patients. J Hum Genet. 2012; 57(5):316-319. doi:10.1038/jhg.2012.24
Chou C C, Zhang Y, Umoh M E, et al. TDP-43 pathology disrupts nuclear pore complexes and nucleocytoplasmic transport in ALS/FTD. Nat Neurosci. 2018; 21(2):228-239. doi:10.1038/s41593-017-0047-3
Corrado L, Ratti A, Gellera C, et al. High frequency of TARDBP gene mutations in Italian patients with amyotrophic lateral sclerosis. Hum Mutat. 2009; 30(4):688-694. doi:10.1002/humu.20950
Dewey C M, Cenik B, Sephton C F, et al. TDP-43 is directed to stress granules by sorbitol, a novel physiological osmotic and oxidative stressor. Mol Cell Biol. 2011; 31(5):1098-1108. doi:10.1128/MCB.01279-10
Dobra I, Pankivskyi S, Samsonova A, Pastre D, Hamon L. Relation Between Stress Granules and Cytoplasmic Protein Aggregates Linked to Neurodegenerative Diseases. Curr Neurol Neurosci Rep. 2018; 18(12):107. doi:10.1007/s11910-018-0914-7
Gasset-Rosa F, Lu S, Yu H, et al. Cytoplasmic TDP-43 De-mixing Independent of Stress Granules Drives Inhibition of Nuclear Import, Loss of Nuclear TDP-43, and Cell Death. Neuron. 2019; 102(2):339-357.e7. doi:10.1016/j.neuron.2019.02.038
Ghoshal N, Smiley J F, DeMaggio A J, et al. A new molecular link between the fibrillar and granulovacuolar lesions of Alzheimer's disease. Am J Pathol. 1999; 155(4):1163-1172. doi:10.1016/S0002-9440(10)65219-4
Goh C W, Lee I C, Sundaram J R, et al. Chronic oxidative stress promotes GADD34-mediated phosphorylation of the TAR DNA-binding protein TDP-43, a modification linked to neurodegeneration. J Biol Chem. 2018; 293(1):163-176. doi:10.1074/jbc.M117.814111
Guo W, Chen Y, Zhou X, et al. An ALS-associated mutation affecting TDP-43 enhances protein aggregation, fibril formation and neurotoxicity. Nat Struct Mol Biol. 2011; 18(7):822-830. Published 2011 Jun. 12. doi:10.1038/nsmb.2053
Hasegawa M, Arai T, Nonaka T, et al. Phosphorylated TDP-43 in frontotemporal lobar degeneration and amyotrophic lateral sclerosis. Ann Neurol. 2008; 64(1):60-70. doi:10.1002/ana.21425
Hicks D A, Cross L L, Williamson R, Rattray M. Endoplasmic Reticulum Stress Signalling Induces Casein Kinase 1-Dependent Formation of Cytosolic TDP-43 Inclusions in Motor Neuron-Like Cells. Neurochem Res. 2020; 45(6):1354-1364. doi:10.1007/s11064-019-02832-2
Hilpert K, Winkler D F, Hancock R E. Peptide arrays on cellulose support: SPOT synthesis, a time and cost efficient method for synthesis of large numbers of peptides in a parallel and addressable fashion. Nat Protoc. 2007; 2(6):1333-1349. doi:10.1038/nprot.2007.160
Hofweber M, Dormann D. Friend or foe-Post-translational modifications as regulators of phase separation and RNP granule dynamics. J Biol Chem. 2019; 294(18):7137-7150. doi:10.1074/jbc.TM118.001189
Iguchi Y, Katsuno M, Takagi S, et al. Oxidative stress induced by glutathione depletion reproduces pathological modifications of TDP-43 linked to TDP-43 proteinopathies. Neurobiol Dis. 2012; 45(3):862-870. doi:10.1016/j.nbd.2011.12.002
Johnson B S, Snead D, Lee J J, McCaffery J M, Shorter J, Gitler A D. TDP-43 is intrinsically aggregation-prone, and amyotrophic lateral sclerosis-linked mutations accelerate aggregation and increase toxicity. J Biol Chem. 2009; 284(30):20329-20339. doi:10.1074/jbc.M109.010264
Josephs K A, Whitwell J L, Knopman D S, et al. Abnormal TDP-43 immunoreactivity in A D modifies clinicopathologic and radiologic phenotype. Neurology. 2008; 70(19 Pt 2):1850-1857. doi:10.1212/01.wnl.0000304041.09418.b1
Josephs K A, Dickson D W. TDP-43 in the olfactory bulb in Alzheimer's disease. Neuropathol Appl Neurobiol. 2016; 42(4):390-393. doi:10.1111/nan.12309
Kametani F, Nonaka T, Suzuki T, et al. Identification of casein kinase-1 phosphorylation sites on TDP-43. Biochem Biophys Res Commun. 2009; 382(2):405-409. doi:10.1016/j.bbrc.2009.03.038
Khalfallah Y, Kuta R, Grasmuck C, Prat A, Durham H D, Vande Velde C. TDP-43 regulation of stress granule dynamics in neurodegenerative disease-relevant cell types. Sci Rep. 2018; 8(1):7551. doi:10.1038/s41598-018-25767-0
Kim H J, Taylor J P. Lost in Transportation: Nucleocytoplasmic Transport Defects in ALS and Other Neurodegenerative Diseases. Neuron. 2017; 96(2):285-297. doi:10.1016/j.neuron.2017.07.029
Kinugasa T, Ozaki S, Hamanaka S, Kudo N. The effects of sciatic nerve axotomy on spinal motoneurons in neonatal Bax-deficient mice. Neurosci Res. 2002; 44(4):439-446. doi:10.1016/s0168-0102(02)00171-2
Lee E K. Post-translational modifications of RNA-binding proteins and their roles in RNA granules. Curr Protein Pept Sci. 2012; 13(4):331-336. doi:10.2174/138920312801619411
Leibiger C, Deisel J, Aufschnaiter A, et al. Endolysosomal pathway activity protects cells from neurotoxic TDP-43. Microb Cell. 2018; 5(4):212-214. doi:10.15698/mic2018.04.627
Liachko N F, McMillan P J, Guthrie C R, Bird T D, Leverenz J B, Kraemer B C. CDC7 inhibition blocks pathological TDP-43 phosphorylation and neurodegeneration. Ann Neurol. 2013; 74(1):39-52. doi:10.1002/ana.23870
Liachko N F, McMillan P J, Strovas T J, et al. The tau tubulin kinases TTBK1/2 promote accumulation of pathological TDP-43. PLoS Genet. 2014; 10(12):e1004803. Published 2014 Dec. 4. doi:10.1371/journal.pgen.1004803
Liu R, Yang G, Nonaka T, Arai T, Jia W, Cynader M S. Reducing TDP-43 aggregation does not prevent its cytotoxicity. Acta Neuropathol Commun. 2013; 1:49. doi:10.1186/2051-5960-1-49
Liu-Yesucevitz L, Bilgutay A, Zhang Y J, et al. Tar DNA binding protein-43 (TDP-43) associates with stress granules: analysis of cultured cells and pathological brain tissue. PLoS One. 2010; 5(10):e13250. Published 2010 Oct. 11. doi:10.1371/journal.pone.0013250
Mackenzie I R, Neumann M, Baborie A, et al. A harmonized classification system for FTLD-TDP pathology. Acta Neuropathol. 2011; 122(1):111-113. doi:10.1007/s00401-011-0845-8
Mackenzie I R, Neumann M. Molecular neuropathology of frontotemporal dementia: insights into disease mechanisms from postmortem studies. J Neurochem. 2016; 138 Suppl 1:54-70. doi:10.1111/jnc.13588
MacLaine N J, Oster B, Bundgaard B, et al. A central role for CK1 in catalyzing phosphorylation of the p53 transactivation domain at serine 20 after HHV-6B viral infection. J Biol Chem. 2008; 283(42):28563-28573. doi:10.1074/jbc.M804433200
Maitland M L, Ratain M J. Terminal ballistics of kinase inhibitors: there are no magic bullets. Ann Intern Med. 2006; 145(9):702-703. doi:10.7326/0003-4819-145-9-200611070-00015
Moisse K, Volkening K, Leystra-Lantz C, Welch I, Hill T, Strong M J. Divergent patterns of cytosolic TDP-43 and neuronal progranulin expression following axotomy: implications for TDP-43 in the physiological response to neuronal injury. Brain Res. 2009; 1249:202-211. doi:10.1016/j.brainres.2008.10.021
Nelson P T, Dickson D W, Trojanowski J Q, et al. Limbic-predominant age-related TDP-43 encephalopathy (LATE): consensus working group report [published correction appears in Brain. 2019 Jul. 1; 142(7):e37]. Brain. 2019; 142(6):1503-1527. doi:10.1093/brain/awz099
Nelson P T, Abner E L, Patel E, et al. The Amygdala as a Locus of Pathologic Misfolding in Neurodegenerative Diseases. J Neuropathol Exp Neurol. 2018; 77(1):2-20. doi:10.1093/jnen/nlx099
Neumann M, Sampathu D M, Kwong L K, et al. Ubiquitinated TDP-43 in frontotemporal lobar degeneration and amyotrophic lateral sclerosis. Science. 2006; 314(5796):130-133. doi:10.1126/science.1134108
Neumann M, Kwong L K, Lee E B, et al. Phosphorylation of S409/410 of TDP-43 is a consistent feature in all sporadic and familial forms of TDP-43 proteinopathies. Acta Neuropathol. 2009; 117(2):137-149. doi:10.1007/s00401-008-0477-9
Nonaka T, Kametani F, Arai T, Akiyama H, Hasegawa M. Truncation and pathogenic mutations facilitate the formation of intracellular aggregates of TDP-43. Hum Mol Genet. 2009; 18(18):3353-3364. doi:10.1093/hmg/ddp275
Nonaka T, Suzuki G, Tanaka Y, et al. Phosphorylation of TAR DNA-binding Protein of 43 kDa (TDP-43) by Truncated Casein Kinase 16 Triggers Mislocalization and Accumulation of TDP-43. J Biol Chem. 2016; 291(11):5473-5483. doi:10.1074/jbc.M115.695379
Origone P, Caponnetto C, Bandettini Di Poggio M, et al. Enlarging clinical spectrum of FALS with TARDBP gene mutations: S393L variant in an Italian family showing phenotypic variability and relevance for genetic counselling. Amyotroph Lateral Scler. 2010; 11(1-2):223-227. doi:10.3109/17482960903165039
Perez D I, Gil C, Martinez A. Protein kinases CK1 and CK2 as new targets for neurodegenerative diseases. Med Res Rev. 2011; 31(6):924-954. doi:10.1002/med.20207
Praline J, Vourch P, Guennoc A M, Veyrat-Durebex C, Corcia P. Co-occurrence of progressive anarthria with an S393L TARDBP mutation and ALS within a family. Amyotroph Lateral Scler. 2012; 13(1):155-157. doi:10.3109/17482968.2011.598168
Prpar Mihevc S, Baralle M, Buratti E, Rogelj B. TDP-43 aggregation mirrors TDP-43 knockdown, affecting the expression levels of a common set of proteins. Sci Rep. 2016; 6:33996. Published 2016 Sep. 26. doi:10.1038/srep33996
Russo A, Scardigli R, La Regina F, et al. Increased cytoplasmic TDP-43 reduces global protein synthesis by interacting with RACKI on polyribosomes. Hum Mol Genet. 2017; 26(8):1407-1418. doi:10.1093/hmg/ddx035
Song H, Kim W, Kim S H, Kim K T. VRK3-mediated nuclear localization of HSP70 prevents glutamate excitotoxicity-induced apoptosis and A R accumulation via enhancement of ERK phosphatase VHR activity. Sci Rep. 2016; 6:38452. doi: 10.1038/srep38452
Sun W, Oppenheim R W. Response of motoneurons to neonatal sciatic nerve axotomy in Bax-knockout mice. Mol Cell Neurosci. 2003; 24(4):875-886. doi:10.1016/s1044-7431(03)00219-7
Ticozzi N, LeClerc A L, van Blitterswijk M, et al. Mutational analysis of TARDBP in neurodegenerative diseases. Neurobiol Aging. 2011; 32(11):2096-2099. doi:10.1016/j.neurobiolaging.2009.11.018
Winton M J, Igaz L M, Wong M M, Kwong L K, Trojanowski J Q, Lee V M. Disturbance of nuclear and cytoplasmic TAR DNA-binding protein (TDP-43) induces disease-like redistribution, sequestration, and aggregate formation. J Biol Chem. 2008; 283(19):13302-13309. doi:10.1074/jbc.M800342200
Wolozin B. Regulated protein aggregation: stress granules and neurodegeneration. Mol Neurodegener. 2012; 7:56. doi:10.1186/1750-1326-7-56
Xiong H L, Wang J Y, Sun Y M, et al. Association between novel TARDBP mutations and Chinese patients with amyotrophic lateral sclerosis. BMC Med Genet. 2010; 11:8. doi:10.1186/1471-2350-11-8
Yamashita T, Hideyama T, Hachiga K, et al. A role for calpain-dependent cleavage of TDP-43 in amyotrophic lateral sclerosis pathology. Nat Commun. 2012; 3:1307. doi:10.1038/ncomms2303
Zhang Y J, Xu Y F, Cook C, et al. Aberrant cleavage of TDP-43 enhances aggregation and cellular toxicity. Proc Natl Acad Sci USA. 2009; 106(18):7607-7612. doi:10.1073/pnas.0900688106
Zhang Y J, Gendron T F, Xu Y F, Ko L W, Yen S H, Petrucelli L. Phosphorylation regulates proteasomal-mediated degradation and solubility of TAR DNA binding protein-43 C-terminal fragments. Mol Neurodegener. 2010; 5:33. Published 2010 Aug. 30. doi:10.1186/1750-1326-5-33

1. A peptide comprising: (a) a cell membrane penetration (CMP) sequence; and (b) one or more TDP-43 phosphorylation-blocking (PB) sequences.

2. The peptide according to claim 1, wherein the PB sequence comprises an amino acid sequence having at least about 90% identity with the amino acid sequence of SEQ ID NO: 10.

3. The peptide according to claim 1, wherein the PB sequence comprises an amino acid sequence having at least about 95% identity with the amino acid sequence of SEQ ID NO: 10.

4. The peptide according to claim 1, wherein the PB sequence comprises the amino acid sequence of SEQ ID NO: 10.

5. The peptide according to claim 1, wherein the PB sequence comprises an amino acid sequence having at least about 90% identity with the amino acid sequence of SEQ ID NO: 11.

6. The peptide according to claim 1, wherein the PB sequence comprises an amino acid sequence having at least about 95% identity with the amino acid sequence of SEQ ID NO: 11.

7. The peptide according to claim 1, wherein the PB sequence comprises the amino acid sequence of SEQ ID NO: 11.

8. The peptide according to claim 1, wherein the CMP sequence comprises an amino acid sequence derived from the HIV-1 Tat domain.

9. The peptide according to claim 8, wherein the peptide comprises an amino acid sequence having at least about 90% identity with the amino acid sequence of one of SEQ ID NO:1, SEQ ID NO: 2, SEQ ID NO:3, SEQ ID NO:4, or SEQ ID NO:5.

10. The peptide according to claim 8, wherein the peptide comprises an amino acid sequence having at least about 95% identity with the amino acid sequence of one of SEQ ID NO:1, SEQ ID NO: 2, SEQ ID NO:3, SEQ ID NO:4, or SEQ ID NO:5.

11. The peptide according to claim 8, wherein the peptide comprises the amino acid sequence of one of SEQ ID NO:1, SEQ ID NO:2, SEQ ID NO:3, SEQ ID NO:4, or SEQ ID NO:5.

12-64. (canceled)

65. A vector encoding a peptide according to claim 1.

66. A polynucleotide encoding a peptide according to claim 1.

67. A pharmaceutical composition comprising:
an effective amount of a peptide according to claim 1, or a pharmaceutically acceptable salt or derivative thereof; and
at least one pharmaceutically acceptable excipient or adjuvant.

68. The peptide according to claim 2, wherein the CMP sequence comprises an amino acid sequence derived from the HIV-1 Tat domain.

69. The peptide according to claim 3, wherein the CMP sequence comprises an amino acid sequence derived from the HIV-1 Tat domain.

70. The peptide according to claim 4, wherein the CMP sequence comprises an amino acid sequence derived from the HIV-1 Tat domain.

71. The peptide according to claim 5, wherein the CMP sequence comprises an amino acid sequence derived from the HIV-1 Tat domain.

72. The peptide according to claim 6, wherein the CMP sequence comprises an amino acid sequence derived from the HIV-1 Tat domain.

73. The peptide according to claim 7, wherein the CMP sequence comprises an amino acid sequence derived from the HIV-1 Tat domain.


SEQUENCE LISTING (11 sequences)

SEQ ID NO: 1 - protein, 25 aa, synthetic construct
YGRKKRRQRRRRIEYIHSKNFIHRD

SEQ ID NO: 2 - protein, 25 aa, synthetic construct
YGRKKRRQRRRTHQHIPYRENKNLT

SEQ ID NO: 3 - protein, 25 aa, synthetic construct
YGRKKRRQRRRSLRFDDKPDYSYLR

SEQ ID NO: 4 - protein, 25 aa, synthetic construct
YGRKKRRQRRRGFSYDYVFDWNMLK

SEQ ID NO: 5 - protein, 32 aa, synthetic construct
YGRKKRRQRRRDGTVLLGGGGFGSSMDSKSSG

SEQ ID NO: 6 - protein, 25 aa, synthetic construct
YGRKKRRQRRRIEHSFRYHRIDNKI

SEQ ID NO: 7 - protein, 14 aa, synthetic construct
RIEYIHSKNFIHRD

SEQ ID NO: 8 - protein, 14 aa, synthetic construct
THQHIPYRENKNLT

SEQ ID NO: 9 - protein, 14 aa, synthetic construct
SLRFDDKPDYSYLR

SEQ ID NO: 10 - protein, 14 aa, synthetic construct
GFSYDYVFDWNMLK

SEQ ID NO: 11 - protein, 21 aa, synthetic construct
DGTVLLGGGGFGSSMDSKSSG